(19) United States
(12) Patent Application Publication
(10) Pub. No.: US 2009/0299141 A1
(43) Pub. Date: Dec. 3, 2009

(54) Laparoscopic Surgical Instrument

(76) Inventors: Earl C. Downey, Salt Lake City, UT (US); Stephen B. Carter, Poway, CA (US); Kenneth A. Gross, Northridge, CA (US); Darrin I. Schmuckle, Vista, CA (US); Robson L. Splane, JR., Valley Center, CA (US)

Correspondence Address: THORPE NORTH & WESTERN, LLP., P.O. Box 1219, SANDY, UT, 84091-1219, US

(21) Appl. No.: 12/430,777

(22) Filed: Apr. 27, 2009

Related U.S. Application Data
(60) Provisional application No. 61/125,536, filed on Apr. 25, 2008.

(51) Int. Cl. A61B 1/00

(52) U.S. Cl. 600/118; 600/131

(57) ABSTRACT

A laparoscopic surgical instrument configured to be ergonomic and anthropometrically correct, comprising: a) an ergonomic handle configured to orient a hand of a surgeon in a functional position, the handle comprising a wall structure defining an interior portion, and adapted to contain at least a portion of one or more working mechanisms; b) an actuating mechanism actuatable by the surgeon and supported within the interior portion of the handle; c) a working shaft having a proximal end coupled to and operable with the actuating mechanism, the working shaft having an elongate configuration and a distal working end configured to couple a surgical tool to be manipulated by the surgeon; and d) means for accessing the interior portion of the handle to expose an inner side of the wall structure and at least a portion of each of the working mechanisms for cleaning, sterilization and maintenance purposes.

### REFERENCE TO RELATED APPLICATIONS

This application claims the benefit of U.S. Application Ser. No. 61/125,536, filed Apr. 25, 2008, and entitled, “Laparoscopic Surgical Instrument,” which is incorporated by reference in its entirety herein.

### FIELD OF THE INVENTION

The present invention relates generally to surgical instruments, and more particularly to working surgical instruments, such as laparoscopic surgical instruments, that provide an internal working mechanism that is actuatable by a user to control the operations of a functional end.

### BACKGROUND OF THE INVENTION AND RELATED ART

Laparoscopic surgical instruments used for laparoscopic surgery vary significantly in design. Many previous designs for laparoscopic instruments are such that their handles are configured so as to orient the hand of a surgeon at a right angle or at substantially a right angle to the instrument and not in a functional and ergonomically correct position. These instruments were designed primarily to allow the surgeon to achieve a direct line of sight through a sheath and into the area where the instrument was intended to perform a surgical task. Because of this, the instruments were awkward and difficult to use for any extended period of time or for lengthy procedures. Moreover, they were not designed for complex internal surgical operations, such as suturing. As such, the function of these instruments largely dictated their form.

Surgical instruments incorporating such designs can be difficult to use, and can also cause injury to the surgeon. The design requires the operator to hold their wrist in awkward positions in order to manipulate the instrument. These positions are not only awkward, but they also encourage the development of carpal tunnel syndrome and chronic joint stress by positioning or orienting the hand in non-natural or non-functional positions, namely at right or substantially right angles relative to the instrument. The problems of joint stress and carpal tunnel are compounded as the surgeon actuates and repeatedly actuates the working mechanisms built into the instruments.

In attempts to alleviate often experienced pain and fatigue that are associated with use of the instrument in its intended manner, particularly in the event of long surgical procedures, many surgeons have resorted to holding the surgical instruments in a manner that is inconsistent with their design. This creates undesirable distractions, delays, and other problems during a surgical procedure.

Additionally, these awkward designs do not efficiently translate force from the handle to the functional end of the instrument. Although the design of the instrument is intended to translate the forces that are applied to the handle to the functional end to perform a desired action, if the handle configured in a manner so as to cause the surgeon's hand to be out of the functional position, a portion of the applied force will be translated to movement of the instrument in a direction that is essentially perpendicular to this axis. This undesirable movement may be translated along the instrument to the functional end, thus compromising stability and inducing unwanted movement.

With the advent of fiber optics, the requirements for current designs have largely eliminated. Rather than using a sheath to facilitate direct line of sight, surgeons today manipulate surgical instruments by means of a camera coupled to the surgical instrument that displays images onto a video screen. Given this change in technology regarding the visual aspects of surgery, it is surprising that the design of laparoscopic surgical instruments has largely remained unchanged. By utilizing modern technology, there no longer is a requirement that traditional or conventional designs be perpetuated. As such, there remains a need for a laparoscopic instrument design that is more ergonomic and anthropometrically correct, simple to use and less strenuous on the surgeon.

Additionally, such laparoscopic instruments are used in surgical environments that are highly susceptible to infectious bacteria and pathogens. During surgery blood and other bodily fluids may come into contact with the instrument and contaminate the handle and other workings. In order to reuse the instrument, it must be thoroughly cleaned and sterilized. Such cleaning is difficult, if not impossible, for the internal workings of the handles that manipulate the surgical instruments. As such, there is an additional need for laparoscopic instruments to provide for cleaning and sterilization of various inner surfaces and for more thorough cleaning and sterilization of working and other mechanisms.

### SUMMARY OF THE INVENTION

In light of the problems and deficiencies inherent in the prior art, the present invention seeks to overcome these by providing a laparoscopic surgical instrument comprising an ergonomic design in combination with a unique trigger control or actuation system. Additionally, the present invention provides a laparoscopic surgical instrument comprising a handle with means for accessing an internal space of the handle and components of the actuation system housed therein.

In accordance with the invention as embodied and broadly described herein, the present invention features a laparoscopic surgical instrument configured to be ergonomic and anthropometrically correct, the laparoscopic surgical instrument comprising: a) an ergonomic handle configured to orient a hand of a surgeon in a functional position, the handle comprising a wall structure defining an interior portion, and adapted to contain at least a portion of one or more working mechanisms; b) an actuating mechanism actuatable by the surgeon and supported within the interior portion of the handle; c) a working shaft having a proximal end coupled to and operable with the actuating mechanism, the working shaft having an elongate configuration and a distal working end configured to couple a surgical tool to be manipulated by the surgeon; and d) means for accessing the interior portion of the handle to expose an inner side of the wall structure and at least a portion of each of the working mechanisms for cleaning, sterilization and maintenance purposes.

The present invention also features a laparoscopic surgical instrument configured to be ergonomic and anthropometrically correct, the laparoscopic surgical instrument comprising: a) an ergonomic handle configured to orient a hand of a surgeon in a functional position, the handle comprising a wall structure defining an interior portion, and adapted to contain at least a portion of one or more working mechanisms; b) an actuating mechanism actuatable by the surgeon and supported within the interior portion of the handle; c) a trigger assembly pivotally supported and operable with the ergonomic handle and the actuating mechanism, the trigger assembly comprising a trigger configured to receive at least one finger of the surgeon and an actuator that operates the actuating mechanism; d) a working shaft having a proximal end coupled to and operable with the actuating mechanism, the working shaft having an elongate configuration and a distal working end configured to couple a surgical tool to be manipulated by the surgeon; and e) a locking mechanism that directly engages and locks the trigger assembly in one of a plurality of positions, the locking mechanism comprising a release located in an anthropometrically correct position.

The present invention also features a laparoscopic surgical instrument configured to be ergonomic and anthropometrically correct, the laparoscopic surgical instrument comprising: a) an ergonomic handle configured to orient a hand of a surgeon in a functional position, the handle comprising a wall structure defining an interior portion, and adapted to contain at least a portion of one or more working mechanisms; b) an actuating mechanism actuatable by the surgeon and supported within the interior portion of the handle; c) a working shaft having a proximal end coupled to and operable with the actuating mechanism, the working shaft having an elongate configuration and a distal working end configured to couple a surgical tool to be manipulated by the surgeon; and d) a first interchangeable trigger assembly pivotally supported and operable with the ergonomic handle and the actuating mechanism, the first trigger assembly being selectively interchangeable with a second trigger assembly having a configuration different from the first trigger assembly.

The present invention further features a method for cleaning and sterilizing of a surgical instrument including obtaining a laparoscopic surgical instrument configured to be ergonomic and anthropometrically correct. The laparoscopic surgical instrument has an ergonomic handle with a wall structure defining an interior portion adapted to contain at least a portion of one or more working mechanisms. The interior portion of the handle can be accessed to expose one or more surfaces of the interior and at least a portion of each of the working mechanisms. The exposed surfaces and the working mechanisms can be cleaned and sterilized to prepare the surgical instrument for subsequent use.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 illustrates a perspective view of an assembled and operable laparoscopic surgical instrument according to one exemplary embodiment of the present invention;

FIG. 2 illustrates a perspective view of the laparoscopic surgical instrument of FIG. 1, as partially exploded;

FIG. 3 illustrates a detailed cut-away perspective view of the various mechanisms and corresponding components of the laparoscopic surgical instrument of FIG. 1;

FIG. 4 illustrates a detailed, exploded perspective view of the reticulation system and the coupling configuration of the working shaft to the handle of the laparoscopic surgical instrument of FIG. 1;

FIG. 5 illustrates a side view of a laparoscopic surgical instrument according to another exemplary embodiment of the present invention, wherein the surgical instrument does not comprise an actuating mechanism;

FIG. 6 illustrates a side view of a laparoscopic surgical instrument according to another exemplary embodiment of the present invention;

FIG. 7-A illustrates a side view of the laparoscopic surgical instrument of FIG. 6, shown with an access opening formed by first and second hinged handle portions in an open position to allow access to an interior portion of the laparoscopic surgical instrument for cleaning and sterilization purposes;

FIG. 7-B illustrates a detailed side view of the laparoscopic surgical instrument of FIG. 6, wherein exemplary embodiments of the various working mechanisms are depicted;

FIG. 8 illustrates a side view of a laparoscopic surgical instrument according to another exemplary embodiment of the present invention;

FIG. 9 illustrates a side view of the laparoscopic surgical instrument of FIG. 8, shown with an access opening in removed from a handle of the laparoscopic surgical instrument to allow access to an interior portion of the laparoscopic surgical instrument for cleaning and sterilization purposes;

FIG. 10 illustrates a perspective view of a laparoscopic surgical instrument in accordance with another exemplary embodiment, shown with a slidable lock on a hinged and pivotal access panel;

FIG. 11 illustrates a side view of the laparoscopic surgical instrument of FIG. 10, with the slidable lock on the access panel in a locked position;

FIG. 12 illustrates a side view of the laparoscopic surgical instrument of FIG. 10, shown with the slidable lock on the access panel in an unlocked position;

FIG. 13 illustrates a side view of the laparoscopic surgical instrument of FIG. 10, shown with the slidable lock unlocked and the access opening pivoted to an open position to allow access to an interior portion of the laparoscopic surgical instrument for cleaning and sterilization purposes;

FIG. 14 illustrates a side view of a laparoscopic surgical instrument according to another exemplary embodiment of the present invention, shown with a first and second interchangeable trigger assembly;

FIG. 15 illustrates a side view of the laparoscopic surgical instrument of FIG. 14, shown with a retaining cap removed to facilitate removal of the first trigger assembly;

FIG. 16 illustrates a side view of the laparoscopic surgical instrument of FIG. 14, shown with an access panel opened to allow access to an interior portion of the laparoscopic surgical instrument for cleaning and sterilization purposes;

FIG. 17 illustrates a perspective view of a trigger assembly in accordance with one exemplary embodiment of the present invention;

FIG. 18 illustrates the trigger assembly of FIG. 17, with the interchangeable portion removed;

FIG. 19 illustrates a perspective view of a trigger assembly in accordance with another exemplary embodiment of the present invention;

FIG. 20 illustrates the trigger assembly of FIG. 19, with the interchangeable portion removed;

FIG. 21 illustrates a perspective view of a trigger assembly in accordance with another exemplary embodiment of the present invention; and

FIG. 22 illustrates a perspective view of the trigger assembly of FIG. 21 with the interchangeable portion detached.

### DETAILED DESCRIPTION OF EXEMPLARY EMBODIMENTS

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

The present invention describes a method and system for providing an ergonomically and anthropometrically correct laparoscopic surgical instrument. In essence, the present invention laparoscopic surgical instrument is intended to be most applicable to the typical working endoscopic instrument. In terms of functionality, the present invention instrument is designed to perform, in an improved manner, the functions or procedures that are most typical in an endoscopic operation, such as cutting tissue, grasping tissue and structures, holding tissues and other objects such as needles, spreading tissues and structures, and so forth. In combination with the handle and the mechanisms operable therewith, the surgical tool located at the distal end of the working shaft of the instrument is capable performing all of these functions, thus making the instrument both versatile and functional. For example, the present invention instrument provides not only the force required to hold strong tissues, but also an improved degree of control, wherein the surgeon is able to sense the degree of pressure being applied.

It is specifically noted that the present invention laparoscopic instrument comprises a “working” instrument that works in both directions allowing closed and open functional movements typically required of a laparoscopic instrument. This is contrary to other types of surgical instruments, such as staplers, coagulation devices, etc. that perform other functions, and that do not require that the instrument function when opening it, for example.

At the outset, the phrase “functional position,” as used herein, shall be understood to mean the well known natural or neutral orientations of the closed or semi-closed hand. One particular functional position of the hand may be identified with the wrist within 20°-30° of extension, the thumb abducted, the metacarpophalangeal joints in 15°-45° flexion, the proximal interphalangeal joints in 25°-30° flexion, and the distal interphalangeal joints in slight flexion.

The phrase “anthropometrically correct,” as used herein, shall be understood to describe various actuating or functional components of the present invention surgical instrument that are located within the measurements of a hand of a surgeon in the substantially functional position, and particularly a surgeon grasping the handle of the surgical instrument, and that are operable by the surgeon in this position.

The phrases “surgery”, “surgical function,” or “surgical procedure,” as used herein, shall be understood to mean any type of activity, action, task, or motion performed by the present invention laparoscopic surgical instrument or the surgical tool coupled thereto. Examples of surgical functions include, but are not limited to, cutting or excision of tissue, clamping or grasping of tissue, and others.

The phrase “surgical tool,” as used herein, shall be understood to mean any type of instrument, device, system, assembly, that attaches or couples to the working end of the working shaft of the present invention laparoscopic surgical instrument capable of performing a surgical function. Examples of surgical tools include, but are not limited to, scissors, excisors, scalpels, clamps, mirrors, lasers, lights, cameras, and others.

The present invention provides several significant advantages over prior related surgical instruments, some of which are recited here and throughout the following more detailed description. First, the present invention laparoscopic surgical instrument provides an ergonomically and anthropometrically correct design that enables the surgeon to orient his or her hand in a functional position, and to operate all mechanisms of the instrument with a single hand with minimal stress and effort. The anthropometric design provides a greater degree of control, thus allowing the surgeon to sense the degree of pressure that is being applied and to know when too much force is being exerted that may cause damage to tissue or surrounding areas. Second, the bi-directional operation and intuitive displacement of the actuating mechanism are well suited for endoscopic operating procedures. Third, the use of a second pinion gear enables both intuitive operation of the actuating mechanism and also a mechanical advantage aspect that provides for increased forces to be applied to the surgical tool through actuation of the trigger. In addition, the added pinion gear enables more delicate and precise movements due to the mechanical advantage. Fourth, the accessibility of the interior portion and working mechanisms contained therein provides for improved cleaning, sterilization and maintenance of the laparoscopic surgical instrument. Fifth, the interchangeability of the trigger assemblies allows the surgeon to customize the surgical instrument according to the surgeon's preferences or to facilitate a particular surgical procedure.

Each of the above-recited advantages will be apparent in light of the detailed description set forth below, with reference to the accompanying drawings. These advantages are not meant to be limiting in any way. Indeed, one skilled in the art will appreciate that other advantages may be realized, other than those specifically recited herein, upon practicing the present invention.

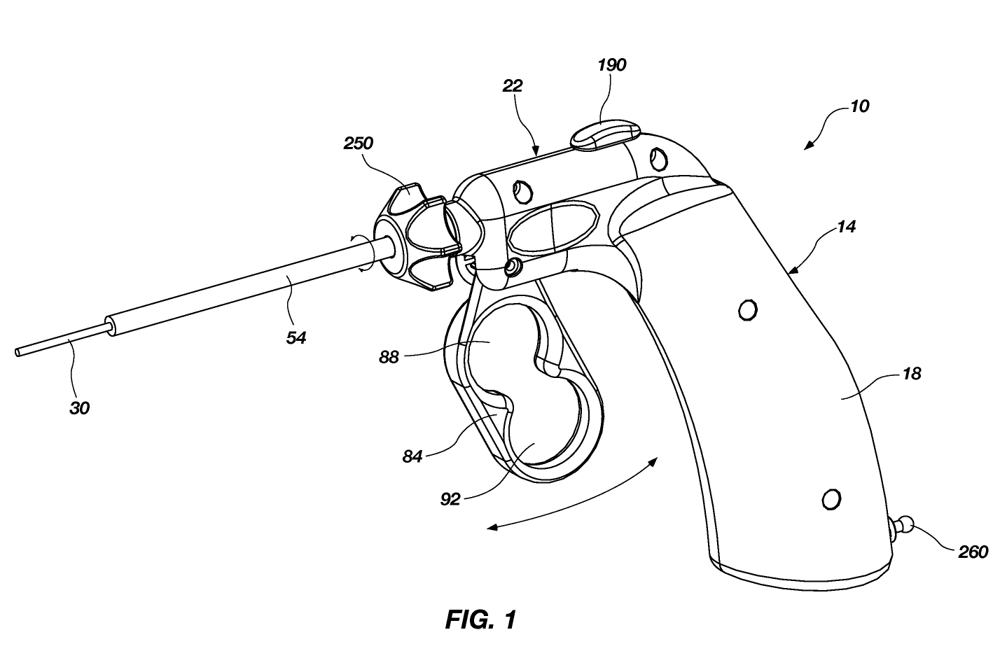

With reference to FIG. 1, illustrated is a perspective view of an assembled and operable laparoscopic surgical instrument according to one exemplary embodiment of the present invention. As shown, the laparoscopic surgical instrument 10 comprises a handle 14 that is configured with anthropometric configuration, as well as being configured to reorient the working axis of the instrument to be in-line concept. This eliminates the up-and-down movement that occurs with prior related surgical instruments utilizing a scissor-type mechanism. In addition, these prior related instruments work the rod, which transmits the actions of the surgeon from the side. On the other hand, the present invention handle design places this action down the center of the handle which may be manipulated in line by the finger of the surgeon, which is held in the functional position because of the orientation of the mechanism along the axis of the handle.

The handle 14 also provides a transfer of the dynamic actuating mechanism to a more central and intrinsic location within the handle itself, as if it really were a part of the hand. This allows a more ergonomic alignment of the actual actuating mechanism of the instrument, reducing stress on joints of the fingers and wrist. The fundamental action used to work a double action or single action surgical tool would remain unchanged. The handle could be configured to detach or rotate into a straight line, leaving a straight grasper/retractor for static functioning with a lower profile that would not tangle light and camera cords, for example.

In the exemplary embodiment shown, the handle 14 comprises a handle grip 18 configured to be grasped by a hand of a surgeon, and a riser 22, configured to extend a portion of the handle 14 away from the hand of the surgeon and to support a working rod or shaft 30 and a sleeve 54 enclosing the working shaft 30. The handle 14 is specifically configured to orient the hand of the surgeon in one or more functional positions, as such positions are commonly understood and/or defined herein, thus providing a more natural and comfortable handle as compared to those existing in the art, as well as reducing the possibility of injury to the surgeon, which injuries may include carpal tunnel syndrome, chronic joint stress and others similar in nature.

The handle grip 18 comprises an ergonomic tubular structure designed to provide significant comfort to the surgeon, as well as to reduce fatigue and other commonly known problems associated with prior related surgical instruments. The handle grip 18 is further configured as a full hand grip that may be configured to extend beyond or below the surgeon's hand a given distance. By extending the handle grip 18 beyond the hand, the bottom of the handle grip 18 may be set on a steady rest of some sort while performing a surgical function. This is particularly useful in lengthy operations in which a certain surgical function requires precise control for an extended period of time. As will be discussed below in greater detail, the handle grip 18 is offset from the riser to provide a handle 14 that orients the surgeons' hand within a range of functional positions.

The handle 14 may be made of any material common to surgical instruments. Preferably, the handle 14 is made of a plastic or lightweight metal material. The handle 14 may further comprise some type of gripping texture formed in the handle surface to provide improved grip of the handle 14. Alternatively, the handle 14 may comprise a rubber or other material gripping element attached or otherwise incorporated into all or a portion of the handle 14.

The laparoscopic surgical instrument 10 further comprises a working shaft 30 configured to couple, and preferably releasably couple, a plurality of interchangeable surgical tools (not shown) to its distal end, and to enable the operation or function of the surgical tool with the handle 14. Essentially, the working shaft 30 is configured to translate the forces from the various components of the handle 14, such as the actuating mechanism discussed below, to the surgical tool to enable the surgical tool to function as intended. The proximal end of the working shaft 30 is supported within the riser 22 of the handle 14, thus allowing the surgeon to manipulate the surgical tool at the site of operation by manipulating the handle 14 or various components or mechanisms or systems thereof. The working shaft 30 comprises an elongate configuration and is designed to be substantially in line with an actuator shaft (not shown) used to couple the working shaft 30, as discussed below. One particular advantage of the present invention is that no part of the handle 14, and namely the hand grip 18, is required to move or displace, in conjunction with the actuating mechanism, when the actuating mechanism is actuated to manipulate and operate the surgical tool coupled to the working shaft 30. This is unlike many prior related surgical tools, namely those based on an angulated scissor-type handle, wherein actuation of the scissor-type handle functions to cause the working shaft to move in an undesirable manner.

The working shaft 30 may be contained within a sleeve 54, as shown, which sleeve functions, among other things, to protect the working shaft 30. More specifically, the sleeve 54 functions to allow the working shaft to freely move in and out in response to trigger motion, and thus manipulate the functional end while at the same time providing for the reticulating function. The sleeve also functions as an insulator. As most instruments provide a cautery function of some kind and need to conduct an electric current to the tissues from the instrument, the sleeve insulates the working shaft so that current is conducted in a controlled or contained manner, thus eliminating random or inadvertent conduction, which may damage surrounding tissues near the working shaft.

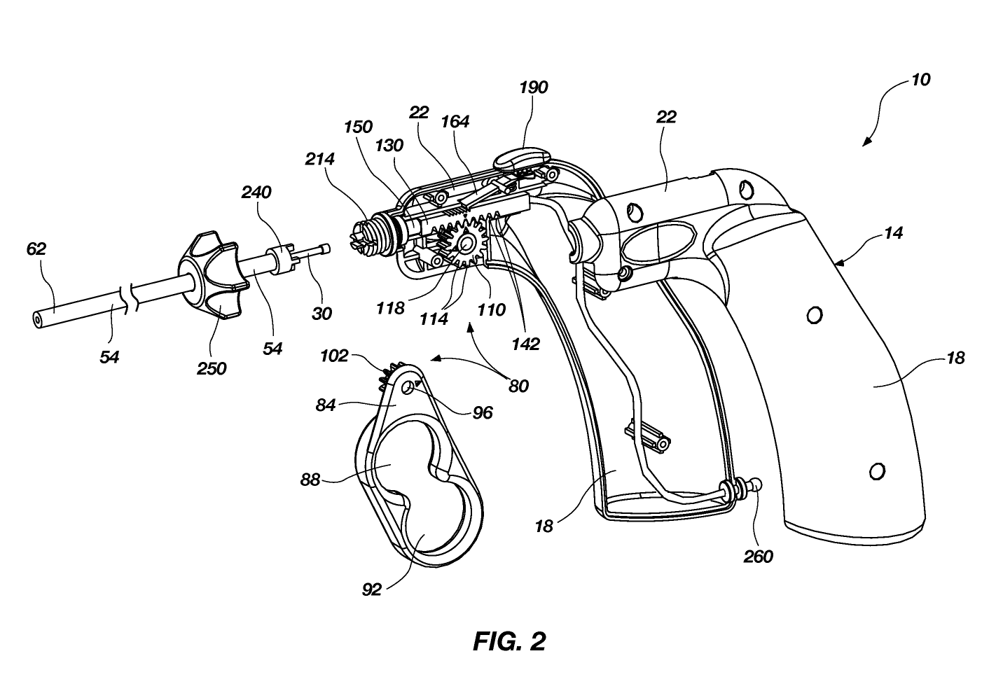

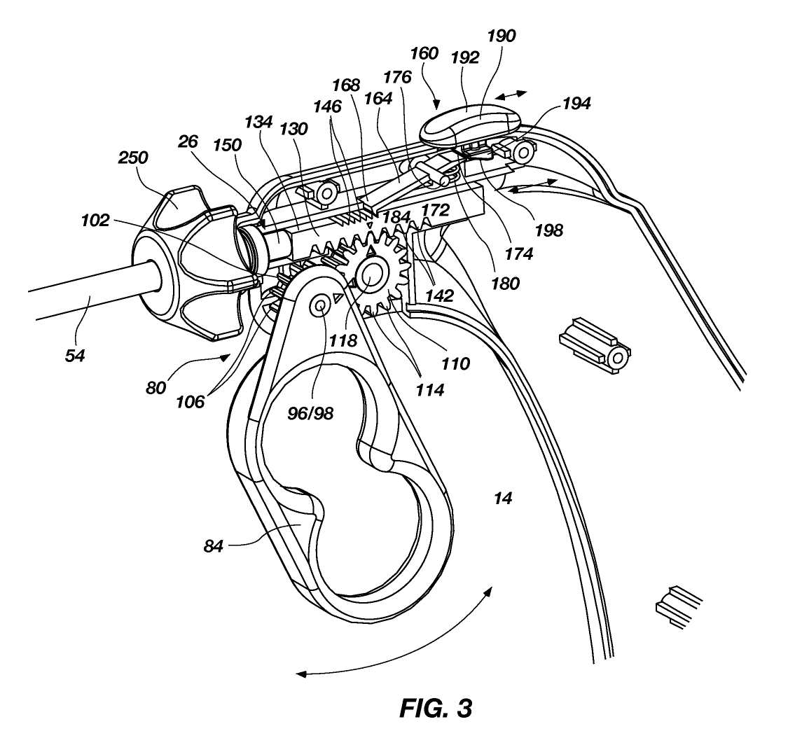

With reference to FIGS. 2 and 3, illustrated are partially exploded perspective views of the laparoscopic surgical instrument of FIG. 1 showing in more detail the internal components of the instrument. The laparoscopic surgical instrument 10 comprises an actuating mechanism 80 configured to manipulate, operate and/or actuate the surgical tool (not shown) coupled to the working shaft 30, depending upon the type of surgical tool being used. The actuating mechanism 80 is supported by the handle 14 and comprises a trigger 84 that extends away from the riser 22 of the handle 14 to be located forward of the handle grip 18, thus placing the trigger 84 in a position to be actuated using the fingers of the hand of the surgeon grasping the handle grip 18. The trigger 84 is rotatably coupled to the riser 22 using a fastener 98. The trigger 84 comprises an axis of rotation 96, or in other words is configured to rotate about pivot point 96. The actuating mechanism further comprises an actuator shaft 130 configured to displace bi-directionally within the riser 22 of the handle 14 upon actuation of the trigger 84. The actuator shaft 30 is configured to operably couple the working shaft 30, such that displacement of the actuator shaft 130 results in a corresponding displacement of the working shaft 30, which functions to control the mechanical function of the surgical tool (e.g., actuate scissors, perform a cutting operation, activate a laser, etc.)

The trigger 84 is shown as comprising first and second finger guides 88 and 92, respectively, configured to receive or accommodate the index and forefingers of the surgeon. The first and second finger guides 88 and 92 are further configured to facilitate bi-directional force from the fingers of the surgeon while simultaneously maintaining thumb, ring, and pinky finger contact on the handle grip 18 to ensure control. In other words, the trigger 84 is configured to receive both forward and backward motion of the fingers of the surgeon. Thus, the trigger 84 comprises supporting structure on both sides of the fingers as inserted into the finger guides 88 and 92. Bi-directional control allows the surgeon to open and close certain styles or types of surgical tools coupled to the working shaft 30.

The trigger 84 may further comprise a soft padded trigger insert configured to improve control of the trigger 84 by minimizing excess space between the inside surface of the finger guides 88 and 92 and the inserted fingers of the surgeon. As such, the trigger insert may be offered in different sizes to be selected by different surgeons of the laparoscopic surgical instrument 10. In addition to providing improved control, the trigger insert functions to improve comfort and reduce fatigue and stress by distributing forces over a larger surface area. This may be particularly useful in lengthy operations to reduce the incidence of cramps, fatigue, soreness or abrasions. The trigger insert may comprise a similar size and shape as the trigger 84, along with the finger guides 88 and 92. In addition, the trigger insert may be configured to removably couple to the trigger 84 using any known fastening means, such as a snap configuration, adhesives, etc.

The actuating mechanism 80 utilizes a gearing system to translate forces from the trigger 84, as applied by the finger(s) of the surgeon, to the working shaft 30, and eventually to the surgical tool attached thereto, thus providing the surgeon with control over the surgical tool attached to the working shaft 30. The gearing system is actuated by displacement or rotation of the trigger 84 about its pivot point 96. The gearing system of the actuating mechanism 80 comprises a pinion gear 102, in the form of an idler spur gear, coupled to or formed on the trigger 84. The pinion gear 102 is located on the trigger 84 such that its axis of rotation is coaxial with the axis of rotation of the trigger 84 about pivot point 96. In other words, the pinion gear 102 shares the same pivot point 96 as the trigger 84. The pinion gear 102 is not a freely rotating gear, but is instead fixed with respect to the trigger 84. Thus, the pinion gear 102 is caused to rotate only upon actuation and resulting rotation of the trigger 84.

The gearing system of the actuating mechanism 80 further comprises a rack and pinion gear combination. A pinion gear 110 is rotatably supported within the riser 22 about pivot point 118. The pinion gear 110 comprises a series of teeth 114 configured to engage and mate with a corresponding rack 142 formed in a lower surface of the actuator shaft 130. The pinion gear 110 and the idler spur pinion gear 102 function together to relate the trigger 84 to the actuator shaft 130, thereby displacing the working shaft 30 to control operation of the surgical too coupled thereto. Indeed, the idler spur pinion gear 102 comprises a series of teeth 106 annularly spaced about its perimeter, which are configured to engage and mate with the teeth 114 formed on the pinion gear 110. Therefore, actuation of the trigger 84 causes idler spur pinion gear 102 to rotate, which in turn induces a corresponding counter rotation in pinion gear 110, which in turn, induces a directional displacement of the actuator shaft 130, which displaces the working shaft 30 to control the surgical tool. The direction of displacement of the actuator shaft 130, and therefore the working shaft 30, is dependent upon the direction in which the trigger 84 is actuated. For example, looking from a point of reference viewing the laparoscopic surgical instrument 10 as oriented as shown in FIGS. 2 and 3, if the trigger 84 is caused to rotate away from the handle grip 18, this causes the idler spur pinion gear 102 to rotate counterclockwise, which induces a clockwise rotation in pinion gear 110. The clockwise rotation of pinion gear 110 causes the actuator shaft 130 and the working shaft 30 coupled thereto to also displace away from the handle grip 18. Conversely, if the trigger 84 is caused to rotate toward the handle grip 18, this causes the idler spur pinion gear 102 to rotate in a clockwise, which induces a corresponding counterclockwise rotation in pinion gear 110. The counterclockwise rotation of pinion gear 110 causes the actuator shaft 130 and the working shaft 30 to also displace toward the handle grip 18. Therefore, unlike many prior related surgical instruments, the present invention laparoscopic surgical instrument 10 provides intuitive operation by movement of the actuator shaft 130 in the same direction as the actuated trigger 84.

Due to the size and configuration of its components, the actuating mechanism 80 further provides a mechanical advantage realized between the trigger 84 and the actuator shaft 130. More specifically, the mechanical advantage enables the trigger 84 to be moved a greater distance relative to the distance that the actuator shaft 130 moves. As such, greater forces may be achieved at the surgical tool, if needed, such as might be the case in cutting, clamping, or grasping tissue. The mechanical advantage provides the surgeon with precise control of the surgical tool, in that large movement of the trigger 84 only results in small movement of the actuator shaft 130, and ultimately the working shaft 30 coupling the surgical tool. Therefore, more delicate procedures requiring greater precision than is available with prior related surgical instruments may be performed.

The mechanical advantage may be different for different instruments. Indeed, the specific mechanical advantage built into a laparoscopic surgical instrument based on the present invention may be varied by operably configuring together different components, such as gear assemblies with different gear ratios. In the exemplary embodiment shown, the mechanical advantage is about 5:1. The mechanical advantage may be configured to be less than or greater than this, but will typically range between 3:1 and 7:1. This range, however, is not to be construed as limiting. For instance, a surgical instrument may be configured with a 1.5:1 or a 10:1 mechanical advantage as well.

Another advantage of the actuation system 80 of the present invention is the minimization of the overall hand motion needed to operate the instrument. Hand motion is indeed minimized as a result of the gearing system employed, in combination with the configuration of the handle to orient the hand in a functional position. Hand motion is further minimized due to the configuration and location of the reticulation system, if employed, which is discussed in greater detail below. By minimizing hand motion, the surgeon is less prone to fatigue and mistakes or injury resulting therefrom.

The present invention laparoscopic surgical instrument 10 further comprises a locking mechanism configured to lock the actuating mechanism 80 in one of a plurality of positions. In the exemplary embodiment shown, the locking mechanism 160 is configured to interact with the actuator shaft 130 to lock the actuating mechanism 80.

More specifically, the locking mechanism 160 comprises a plurality of notches 146 formed on at least a portion of an upper surface 134 of the actuator shaft 130. A pawl 164 having a first end 168 and a second end 172 is configured to engage the notches 146 to lock the actuator shaft 130 in place and to prevent its further displacement. The notches 146 and the pawl 164 are configured to provide a ratcheting effect so that the actuator shaft 130 is capable of moving in a unidirectional manner when the pawl 164 is engaged with the actuator shaft 130. In the exemplary embodiment shown, the notches 146 and pawl 164 are each configured so that, when engaged, the actuator shaft 130 may displace toward the handle grip 18 upon squeezing the trigger 84 to displace it toward the handle grip 18. In this configuration, and with the pawl 164 engaged, the trigger 84 and actuator shaft 130 are prohibited from displacing away from the handle grip 18, thus locking them in place, as well as the working shaft 30 and any components of the surgical tool operable therewith. Other configurations are contemplated herein.

The pawl 164 may be pivotally mounted to a portion of the riser 22 and may be biased by a biasing element 180 toward an engaged position with the notches 146 formed in the actuator shaft 130. The biasing element may comprise any commonly known in the art, and is shown as preferably comprising a spring situated between a support and the lower surface of the second end 172 pawl 164. The pawl 164 is configured to pivot about pivot point 176 with the first end 168 being on one side of the fulcrum support and the second end 172 being on the opposite side. In other words, the pawl 164 is configured to teeter about the fulcrum pivot point 176.

The locking mechanism further comprises a release 190 configured to selectively release the pawl 164 from the actuator shaft 130, thus enabling the actuating mechanism 80 to move in the direction previously prohibited. The release 190 may comprise any type of release, but is preferably a quick release located in an anthropometrically correct position about the handle 14. In the exemplary embodiment shown, the release 190 comprises a thumb release located atop the riser 22. The thumb release 190 comprises an actuator 194 extending down from a button 192. The actuator 194 comprises an inclined surface 198 that is configured to engage a corresponding inclined surface 174 formed in the second end 172 of the pawl 164. In this configuration, the release 190 may be actuated by sliding the button 192 in a forward direction towards the working shaft 130. By displacing the button 192 in this direction, the actuator 194, and particularly its inclined surface 198, slides along the inclined surface 174 of the pawl 164, which causes the pawl 164 to rotate counterclockwise about the pivot point 176. The counterclockwise rotation of the pawl 164 effectively functions to overcome the biasing element 180, thus disengaging the pawl 164 from the actuator shaft 130 and allowing the actuating mechanism 80, namely the trigger 84, the pinion gears 102 and 110, and the actuator shaft 130, to move in the direction previously prohibited. This effectively allows the working shaft 30 to also move in the direction previously prohibited, to control the surgical tool as needed. By actuating the thumb release 190, the actuating mechanism 80 is allowed to displace in bi-directionally. The thumb release therefore functions to override the locking mechanism 160 when it is desired to do so.

The present invention contemplates other types of locking mechanisms for locking the actuating mechanism 80 in place. For example, rather than interacting with and locking the actuator shaft 130, thereby locking the remaining components of the actuating mechanism 80, the locking mechanism may be configured to interact with one of the pinion gears 102 and 110, or the trigger 84 itself.

The locking mechanism 160 functions to provide a variable position lock on the actuating mechanism and works in conjunction with the actuating mechanism 80 and its mechanical advantage. For example, the actuating mechanism 80 allows the surgeon to grasp or clamp an object using a significant amount of force and to lock the actuating mechanism in that position for any period of time. This allows the surgeon to relax his or her grip on the handle 14, while maintaining suitably strong forces on the object being grasped or clamped. As such, the surgeon is able to reduce stresses in the hand and to better concentrate on the operating procedure. In addition, the release 190 is positioned both ergonomically and anthropometrically, allowing the surgeon to actuate the release 190 with one hand while still grasping the handle 14 and actuating the trigger 84.

With reference to FIGS. 2-4, the exemplary laparoscopic surgical instrument 10 further comprises a reticulation system 210 operable with the working shaft 30 and configured to facilitate selective rotation of the working shaft 30 (and/or the sleeve 54 enclosing the working shaft 30) and the surgical tool attached thereto. Specifically, the reticulation system 210 comprises a threaded bushing or threaded collar 214 fittable over and rotatable about a portion of the actuator shaft 130, shown as shaft extension 150. The threaded collar 214 comprises a threaded body 218 juxtaposed to and extending from a flange 226. The threaded collar 214 is supported by the riser 22 via a retaining member 234 configured to be seated within a corresponding groove 26 formed in the riser 22. The retaining member 234 may also configured to rotate within the groove 26 to enable the threaded body portion 218 to rotate, or the retaining member 234 may be seated in a fixed manner within the groove 26 and a portion thereof rotatably coupled to the threaded body 218. The threaded body 218 further comprises one or more keyholes 230 formed therein.

As discussed above, the working shaft 30 may be enclosed or encased within a sleeve, shown as sleeve 54, which sleeve may be coupled to the surgical tool along with the working shaft 30. The sleeve 54 is shown as comprising an elongate body having a proximal end 58 and a distal end 62. The proximal end 58 further comprises a key 240 having one or more key segments 244 configured to engage and mate with the key holes 230 formed in the threaded body 218 of the threaded collar 214. As such, rotation of the threaded collar 214 will induce a corresponding rotation within the sleeve 54, and thus the surgical tool coupled thereto.

To facilitate rotation of the sleeve 54 and ultimately the surgical tool coupled thereto, the reticulation system further comprises a reticulation knob 250 having a threaded bore configured to be threaded onto the threaded body 218 of the threaded collar 214 to nest against the flange 226, thereby securing the reticulation knob 250 to the collar 214. At many times during a surgical procedure there is a necessity to manipulate the surgical tool into several different orientations and positions. The reticulation system is designed to facilitate, via the reticulation knob 250, the easy, efficient, and comfortable rotation of the surgical tool. It is specifically noted herein that the reticulation knob 250 is located in both an ergonomic and anthropometrically correct position, within the reach of a finger of the surgeon, particularly the forefinger. As such, the surgeon can operate the laparoscopic surgical instrument 10 with one hand, which does not have to release the handle grip 18 to rotate the reticulation knob 250. The reticulation system may also be configured to operate electronically, such as via battery power.

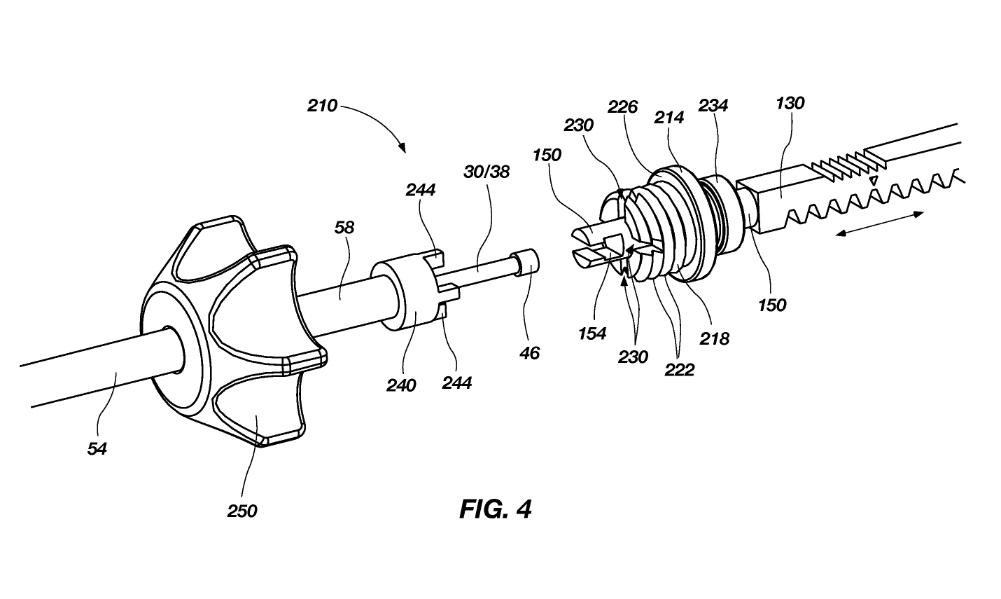

FIG. 4 further illustrates a way of coupling the working shaft 30 to the handle 14, and particularly the actuator shaft 130 contained within the handle 14. This coupling configuration provides many advantages over prior related surgical instruments, namely ease of use and interchangeability. As shown, the shaft extension 150 of the actuator shaft 130 comprises, at its distal end, a coupler 154 configured to receive and couple the proximal end 38 of the working shaft 30, which has located thereon a disc or flange 46 configured to engage and seat within the coupler 154. To couple the working shaft 30 to the actuator shaft 130, the flange 46 is inserted into the coupler 154 through a slotted portion, thereby securing the rim of the flange 46 against the edge of the coupler 154. Once the flange 46 of the working shaft 30 is inserted into and seated within the coupler 154, the key 240 of the sleeve 54 is caused to engage the threaded body 218 of the collar 214. The reticulation knob 250 is then screwed in place, thus securing the coupling connection between the working shaft 30 and the actuator shaft 130. This connection configuration provides for easy interchangeability in that several different types of working shafts 30, each configured to perform a different function, may be easily and quickly interchanged with one another. In other words, several different types of working shafts may be interchanged with one another and used with a single handle, namely handle 14. To uncouple the working shaft 30, the reticulation knob 250 is simply removed, thus allowing the key 240 to disengage from the collar 214. The flange 46 may then be slid out of the coupler 154 through the slotted portion.

Referring back to FIG. 1, the laparoscopic surgical instrument 10 further comprises an electrical connector 260 supported within the handle 14. The electrical connector may be used for various purposes, such as electro-cautery functions. The electrical connector 260 is preferably located on the side of the handle grip 218 near its bottom, thus minimizing the chance for cords to interfere with one another, as well as to reduce the chance of the cords putting undesirable tension on the handle in a manner that would interfere with the proper operation of the surgical instrument.

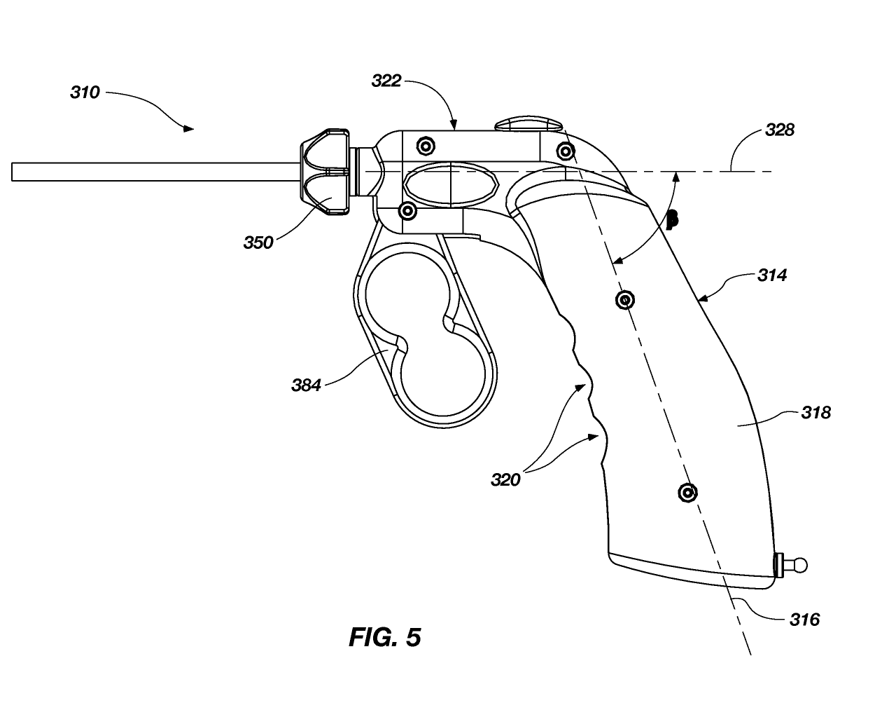

With reference to FIG. 5, illustrated is a side view of a laparoscopic surgical instrument according to another exemplary embodiment of the present invention. In this embodiment, the laparoscopic surgical instrument 310 comprises a similar handle 314 as the one discussed above, as well as an actuation mechanism with trigger 384. As such, the description above is incorporated herein, where applicable. However, unlike the laparoscopic surgical instrument discussed above and illustrated in FIGS. 1-4, the laparoscopic surgical instrument 310 comprises an actuating mechanism with a lesser mechanical advantage that allows the surgeon to perform a surgical procedure where greater force may cause damage to delicate tissue. For example, this particular laparoscopic surgical instrument may be particularly suited for a bowel grasping procedure. By reducing the mechanical advantage, the working end is capable of providing more sensitive operations, while still providing force multiplication. As such, the present invention contemplates laparoscopic surgical instruments with different mechanical advantages to suit different surgical needs.

FIG. 5 also illustrates the laparoscopic surgical instrument 310 as comprising a handle 314 having a handle grip 318 and a riser 322, wherein a working shaft 330 is supported by the riser 322 in a similar manner as discussed above. The laparoscopic surgical instrument 310 also comprises a reticulation system similar to the one discussed above, which is configured to provide rotation to the surgical tool via the reticulation knob 350.

FIG. 5 further illustrates the orientation of the handle grip 318 with respect to the riser 322. As shown, the handle grip 318 comprises a longitudinal axis 316 that is offset a pre-determined angle from a longitudinal axis 328 of the riser 322. The angle β existing between these two axis may be between 60 and 80 degrees (or between 100 and 120 degrees as measured from the working shaft), thus orienting the hand of the surgeon in a functional position. In the embodiment shown, the angle β is 68° (or 112° as measured from the working shaft). The relationship of the handle grip 318 to the riser 322 shown in FIG. 5 and discussed herein is also applicable to the handle 14 discussed above and shown in FIGS. 1-4.

The handle grip 318 may further comprise one or more finger guides formed therein, shown as finger guides 320, as commonly known in the art. These may assist the surgeon in maintaining a proper grip on the handle 314.

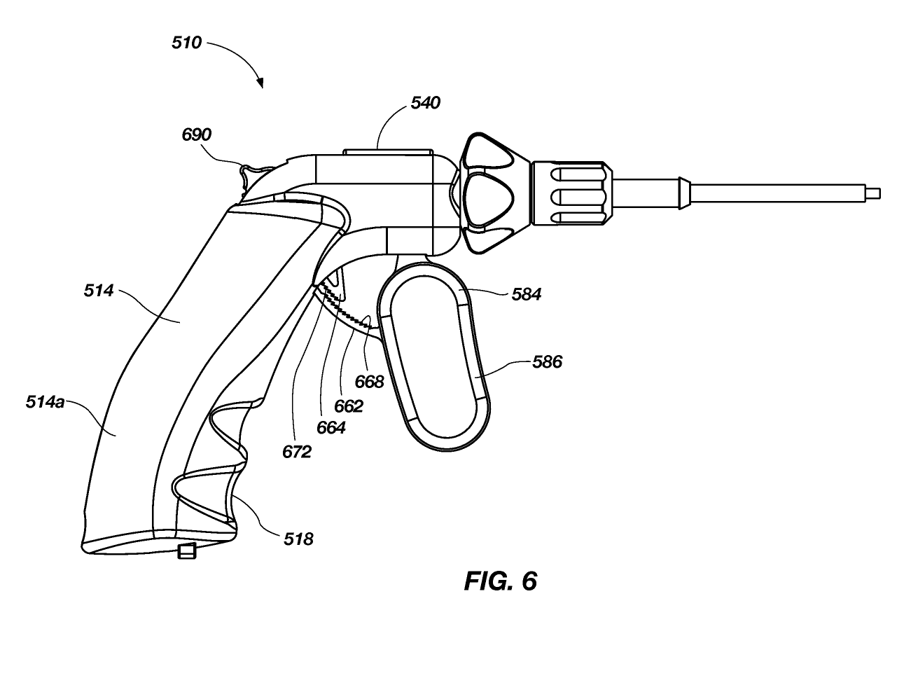

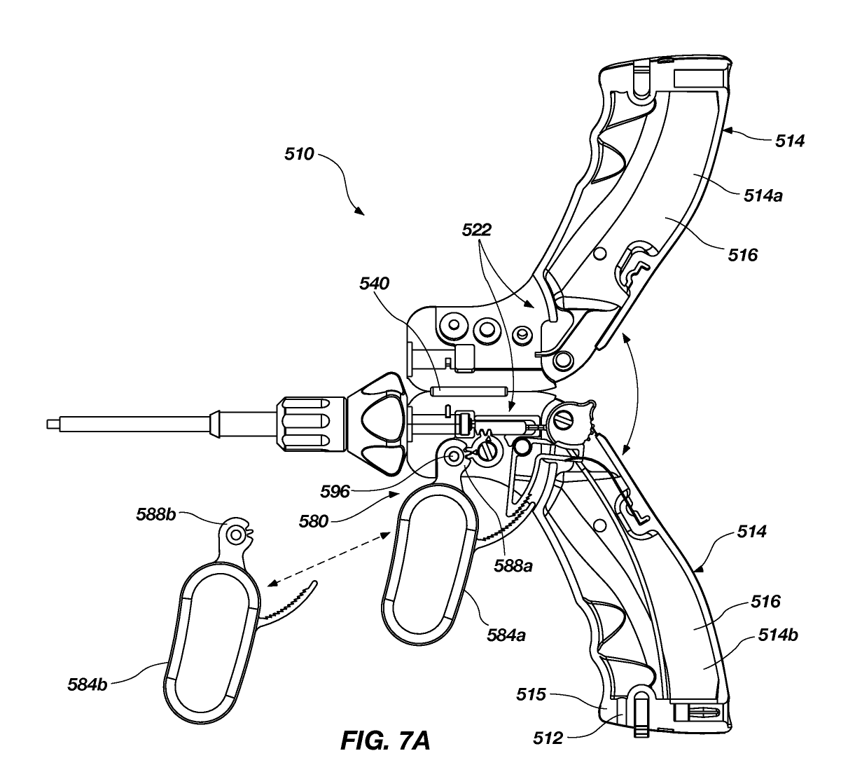

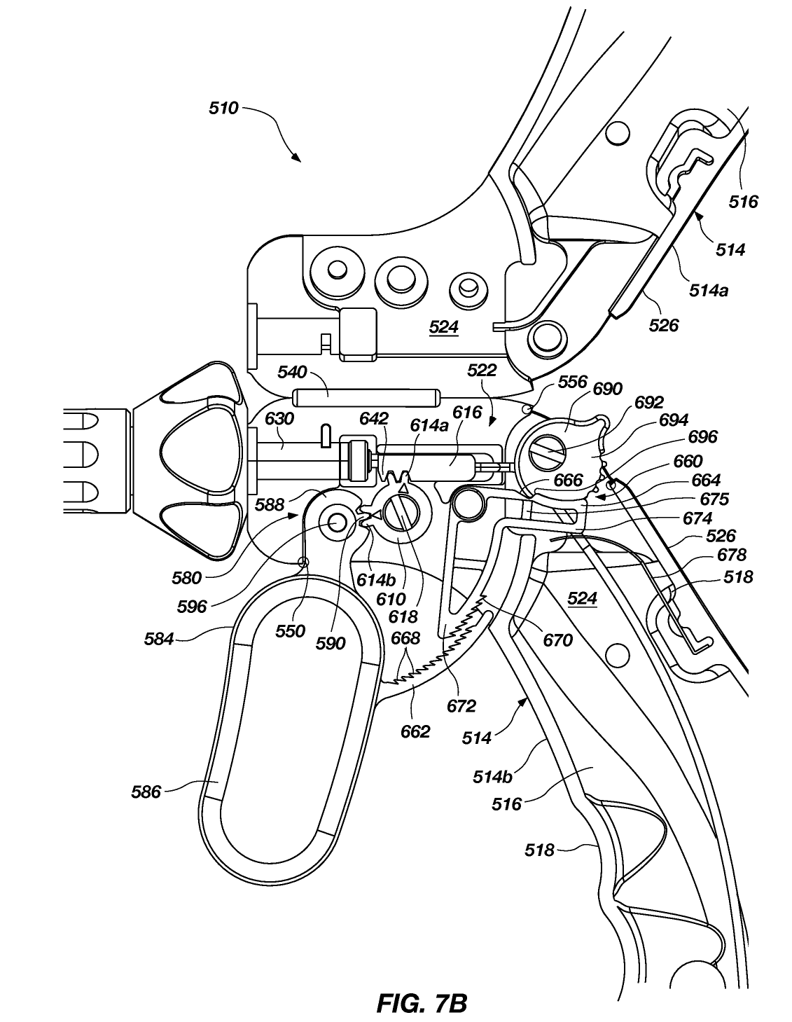

With reference to FIGS. 6-7 illustrated are various side views of a laparoscopic surgical instrument according to another exemplary embodiment of the present invention. In this embodiment, the laparoscopic surgical instrument 510 comprises a similar handle 514 as described above having a wall structure 516 defining an interior portion 522 that at least partially contains and supports one or more working mechanisms, such as an actuating mechanism 580 with a trigger assembly 584 and locking mechanism 660. As such, the description above is incorporated herein, where applicable. However, unlike the laparoscopic surgical instrument discussed above and illustrated in FIGS. 1-5, the laparoscopic surgical instrument 510 comprises an ergonomic handle 514 having a wall structure 516 defining an interior portion 522 containing working mechanisms with fewer tight areas between components of the working mechanisms and other narrow channels that are difficult to clean and sterilize, and which may harbor harmful bacteria and pathogens.

Accordingly, the interior portion 522 can at least partially contain and support one or more working mechanisms such as the actuating mechanism 580, a locking mechanism 660, a working shaft 630, a conducting rod, a gear system, an actuator shaft, a trigger hinge, and combinations of these working mechanisms. For example, the interior portion can contain and support the actuating mechanism 580, a portion of the trigger 584, and a portion of the working shaft 630. In this way, within the interior portion 522 of the handle, the gearing system of the actuating mechanism 580 translates forces from the trigger 584, as applied by the finger(s) of the surgeon, to the working shaft 630, and eventually to the surgical tool attached to a distal end of the working shaft 630, which is outside of the interior portion 522, to provide the surgeon with control over the surgical tool attached to the working shaft 630. Thus, the gearing system is actuated by displacement or rotation of the trigger assembly 584 about its pivot point 596.

More specifically, the trigger assembly 584 includes a trigger 586 coupled to an actuator 588. The trigger 586 is sized and shaped to receive at least one finger of the surgeon. The actuator 588 is a toothed gear that actuates the gearing system of the actuating mechanism 580. The actuator 588 is rotatably coupled to the handle 514 at the pivot point 596 about which the actuator 588 rotates to actuate the gearing system of the actuating mechanism.

Additionally, the gearing system of the actuating mechanism 580 includes a rack and pinion gear combination. A pinion gear 610 is rotatably supported about pivot point 618 within an interior space 622 of the ergonomic handle 514. The pinion gear 610 comprises two sets of teeth 614a and 614b positioned at an approximate 90 degree angular orientation from one another on the pinion gear. The first set of teeth 614a engage and mate with a corresponding rack 642 associated with a lower surface of a collar 616 coupled to the actuator shaft 630. The second set of teeth 614b engage and mate with the teeth 590 in the corresponding actuator gear 588 in the trigger assembly 584. The pinion gear 610 and the trigger actuator gear 588 function together to relate the trigger 584 to the working shaft 630, thereby displacing the working shaft 630, as described above, to control operation of the surgical tool coupled thereto.

The ergonomic handle 514 also includes means for accessing the interior portion 522 of the handle 514 to expose an inner side or surface 524 of the wall structure and at least a portion of each of the working mechanisms for cleaning, sterilization and maintenance purposes. For example, an access opening defined by an outer perimeter boundary 526 of the handle 514 can allow access to the interior portion 522 of the handle 514.

More specifically, in one aspect, the access opening can be formed by an openable, two-piece handle configuration (see FIG. 7-A). In this case, a first handle portion 514a having a perimeter boundary is operable with a second handle portion 514b to define the handle 514. The first and second handle portions 514a and 514b, also having a perimeter boundary, are separable from one another to provide access to the interior portion 522. A hinge 540 is operable to interrelate the first and second handle portions 514a and 514b, and to facilitate selective separation, alignment and union of the first and second handle portions. The hinge 540 pivotally couples and aligns the first and second handle portions 514a and 514b to ensure easy opening and closing of the first and second handle portions, as indicated by the arrows. In one aspect, the hinge may be located about the riser of the handle, as shown. In another aspect, the hinge may be positioned along the back of the grip position to facilitate a different openable configuration.

Advantageously, with the first and second handle portions 514a and 514b in the open configuration, as shown in FIGS. 7-A and 7-B, the interior space 522 and working mechanisms contained therein are selectively exposed and can be easily cleaned, maintained and sterilized. Additionally, with the first and second handle portions 514a and 514b in the closed position, as shown in FIG. 6, the interior portion 522 of the handle 514 is sealed off from the surrounding environment in order to reduce contamination of the working mechanisms and the inside portion of the handle 514. One or more seals may be provided for sealing the first and second handle portions. For example, an elastomeric seal may be located and supported about the perimeter edges of each of the handle portions 514a and 514b, such that when the handle portions are brought together and closed, the seal functions to seal the two portions together. The two piece handle configuration is an example of one means for accessing the interior portion 522 of the handle 514.

Alternatively, rather than using a hinge design, the first and second handle portions 514a and 514b may be completely separable from one another and removably coupled using one or more types of fastening means as known in the art.

Referring again to FIGS. 6-7, the laparoscopic surgical instrument 510 also includes a locking mechanism 660 that directly engages and locks the trigger assembly 584 in one of a plurality of positions. The locking mechanism 660 comprises a first link 662 operable with the trigger assembly 584 and a second link 664 operable with the release 690. The first link 662 has a plurality of teeth 668 formed on at least a portion thereof. The first link 662 is curved and extends away from the trigger assembly 584 toward the grip 518 of the handle 514.

The second link 664 is pivotally mounted to the handle 514 and has at least one tooth 670 formed on at least a portion of said second link 664. The second link 664 comprises a first end 672 and a second end 674. The first end 672 has the at least one tooth 670, and the second end 674 is oriented substantially perpendicular to the first end 672 so that together the two ends form a right angled corner with a pivot 676 between the two ends. The first end 672 has a curve configuration that corresponds substantially to the curve configuration of the first link 662. The second end 674 extends rearward toward the thumb actuated release 690. The at least one tooth 670 of the second link 664 engages the plurality of teeth 668 on the first link 662 to selectively lock the trigger assembly 584 in one of the plurality of positions. The first and second links 662 and 664 provide a ratcheting function that allows the actuating mechanism 580 to move freely in one direction, while being restricted to move in an opposite direction. The first and second links 662 and 664 are selectively releasable from one another upon actuation of the release 690 by the user.

A biasing element 678 biases the second link 664 about the pivot point 676 to an engaged position with respect to the first link 662, such that at least one of the plurality of teeth 670 of the first end 672 of the second link 664 has a tendency to engage at least one of the plurality of teeth 668 on the first link 662, thus locating and locking the trigger in a selected position. In one aspect, as shown, the biasing mechanism 678 comprises a linear spring coupled to the handle 514 in a pre-stressed configuration and is held in place in the interior space 522 by a plurality of mounts 518 formed in the handle.

The thumb actuated release 690 is supported by the handle 514 and engages the second link 664 to overcome the force of the biasing element 678 in order to disengage the second link 664 from the first link 662. In this way, the trigger assembly 584 is enabled to freely move in the opposite direction. To accomplish this, the release 690 is rotatably mounted about a pivot point 692 formed in the ergonomic handle 514. The release 690 comprises an eccentric actuator 694 configured to engage the second end 674 of the second link 664 when rotated by the surgeon. By rotating the release 690 in a given direction the eccentric actuator 694 engages the second end 674 of the second link 664 to pivot the second link about the pivot point 692, thus causing the second link 664 to disengage from the plurality of teeth 668 of the first link 662 to allow the trigger assembly 584 to freely move in any direction. Advantageously, the thumb actuated release 690 is located in an ergonomic and anthropometrically correct position on the handle 514 and namely the upper section of the grip portion of the handle to enable the surgeon to selectively operate the release 690, the trigger 584, and the actuating mechanism 580 simultaneously with the same hand, while maintaining the hand in a substantially functional and anthropometrically correct position. In other words, during operation of the laparoscopic surgical instrument, namely either operation of the trigger in either direction or the thumb actuated release, the hand of the surgeon is maintained in a comfortable position with the handle and the working mechanisms being configured so as to minimize the required flexion of the wrist and fingers in order to achieve normal operation. The surgeon's hand is allowed to be in a more functional and anthropometrically correct position rather than at a right angle or at a substantially right angle relative to the instrument.

The eccentric actuator 694 can also include a protrusion 696. The protrusion 696 is designed to be received within and operable with a detent 666 formed in the upper surface of the second end 674 of the second link 664. Adjacent the detent 666, the upper surface may comprise a curved configuration or curved portion corresponding to the radius of curvature of the outer lower surface of the actuator 694. As can be seen, the actuator 694 is nested within the curved portion of the upper surface of the second end of the second link. As the thumb actuated release 690 and eccentric actuator 694 are actuated, the protrusion 696 initially functions or operates within the detent 666 to apply a load to the second end 674 of the second link 664 to cause the second link 664 to pivot about the pivot point 676. As this cam action continues, the protrusion releases from the detent 666 with the actuator 694 rotating about and driving downward the second link 664, which functions to further overcome the biasing forces applied on the second link 664 by the biasing member 678, and which functions to rotate the second link in a clockwise manner to release the second link 664 from the first link 662. A cross member 675 can support the detent 666 on the second link 664 so that the second end 674 of the second link 664 does not deform under the loading from the protrusion 696 on the eccentric actuator 694. Advantageously, the thumb actuated release 690 may be maintained in this or another position to permit the trigger to pivot freely in both directions to operate a surgical instrument located about the working end of the laparoscopic instrument. Alternatively, the thumb actuated release 690 may be moved into a position such that the ratcheting function discussed above is actuated allowing the trigger to be locked in one of a plurality of positions.

Sealing members, such as silicone boots, o-rings, and the like, can extend around or otherwise be operable with any working mechanisms that extend through the wall structure of the handle in order to reduce the chance of contamination from contaminants entering the interior portion 522 of the handle 514. For example, a trigger seal 550 can extend around the trigger assembly 584 and can seal an aperture (not shown) in the handle 514 through which the trigger assembly 584 extends. Similarly, a release seal 556 can extend around the thumb actuated release 690 and can seal an aperture (not shown) in the handle 514 through which the lock release 690 extends. Other seals can also be used to seal apertures or holes in the handle 514. It is contemplated that any portion of the handle and/or working mechanisms may be sealed, or made to operate with a seal, as needed.

A drain hole 512 (see FIG. 7-A) can be formed in a bottom 515 of the handle 514. The drain hole 512 can be openable to allow fluid to enter or exit the handle 514 during the cleaning process. In this way, cleaning fluid such as pressurized steam can enter the handle 514 and equalize the pressure in the interior portion 522 with the outside environment in order to prevent the handle 514 from being crushed by steam pressure during the cleaning process. In addition, the drain hole 512 may be configured to enable or facilitate passive evaporation of residual moisture or active drying.

With reference to FIGS. 8-9 illustrated are various side views of a laparoscopic surgical instrument according to another exemplary embodiment of the present invention. In this embodiment, the laparoscopic surgical instrument 710 comprises a similar handle 714 as described above having a wall structure defining an interior portion 722 that contains and supports various working mechanisms, such as an actuating mechanism 780 with a trigger 784 and locking mechanism 760. The working mechanisms may comprise a similar design and functionality as any of the preceding embodiments described above. As such, the description above is incorporated herein, where applicable.

The ergonomic handle 714 also includes means for accessing the interior portion 722 to expose an inner side or surface 724 of the wall structure and at least a portion of each of the working mechanisms for cleaning, sterilization and maintenance purposes. Unlike any of the preceding embodiments, in the embodiment shown in FIGS. 8 and 9, means for accessing the interior portion comprises an access opening comprising a removable access panel 790 removably coupled to the wall structure of the ergonomic handle 714. The removable access panel 790 defines and forms an access opening and is positioned so as to cover and selectively expose the actuating mechanism 780 and the locking mechanism 760. The access panel 790 can cover and seal the working mechanisms in the interior portion 722 to protect them from dirt and contamination. The access panel 790 is selectively removable from the wall structure of the handle 714 to provide access to the interior portion 722 containing the working mechanisms and the inner surfaces of the handle. The access panel 790 can be coupled to the handle 714 by fasteners 792, as known in the art, and is shown as comprising a structural configuration that nests or fits between permanent handle portions 792 and 794. The removable access panel 790 is an example of another means for accessing the interior portion 722 of the handle 714. As with the embodiment shown in FIGS. 6-7, one or more seals may be provided that seal the removable access panel 790 once in position about the handle 714. In addition, seals may be provided about any portion of any of the working mechanisms extending outward from the handle 714.

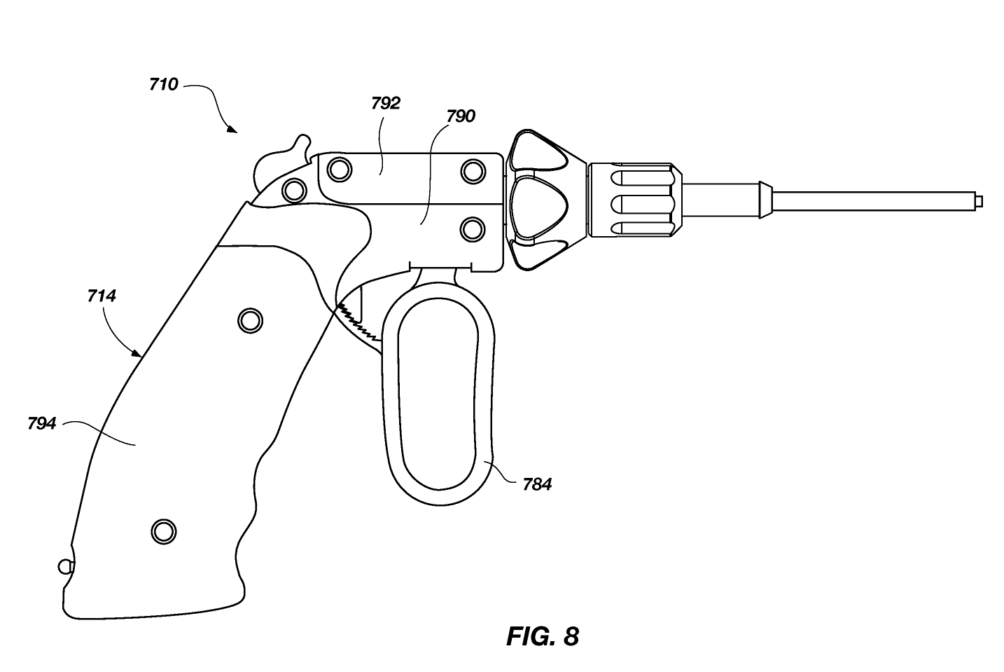

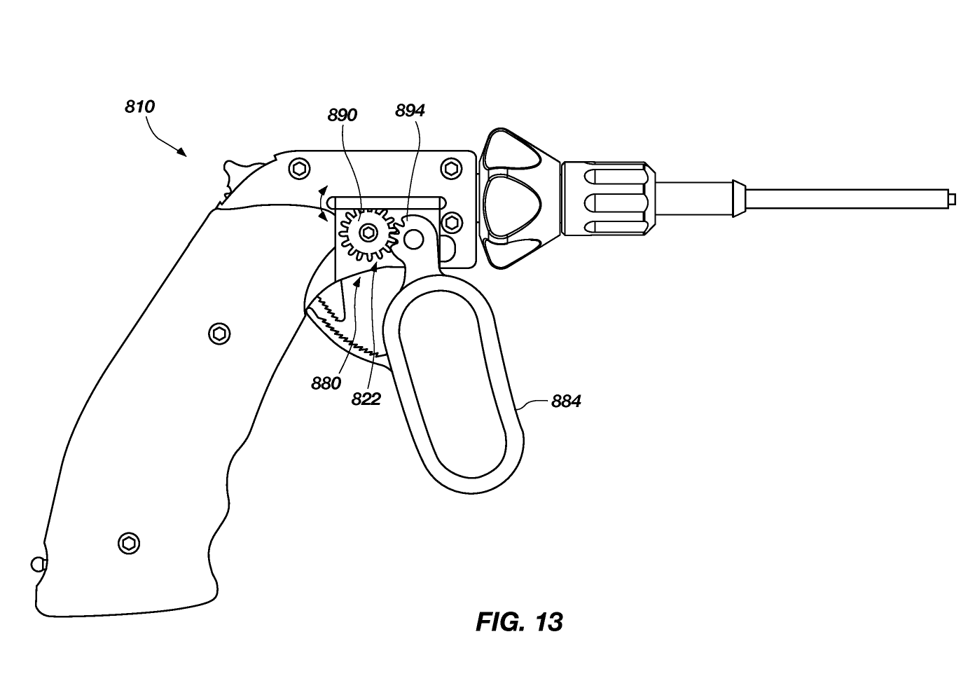

With reference to FIGS. 10-13 illustrated are various views of a laparoscopic surgical instrument according to another exemplary embodiment of the present invention. In this embodiment, the laparoscopic surgical instrument 810 comprises a similar handle 814 as described above and having a wall structure defining an interior portion 822 that contains and supports working mechanisms, such as an actuating mechanism 880 with a trigger 884 and a locking mechanism 860. The working mechanisms may comprise a similar design and functionality as any of the preceding embodiments described above. As such, the description above is incorporated herein, where applicable. However, unlike the laparoscopic surgical instruments discussed above, the laparoscopic surgical instrument 810 comprises an ergonomic handle 814 with a wall structure defining an interior portion 822 having a hinged access panel 890 pivotally coupled to the wall structure of the ergonomic handle 814.

The hinged access panel 890 is hingedly or pivotally coupled to the wall structure of the handle 814 and positioned so as to provide coverage over and selective exposure to the actuating mechanism 880 and the locking mechanism 860 when closed and opened, respectively. The hinged access panel 890 covers the working mechanisms in the interior portion 822 to protect them from dirt and contamination, yet provides simple and easy opening to expose the working mechanisms for cleaning and sterilizing purposes. The access panel 890 is pivotal about a hinge 892 to provide access to the interior portion 822 containing the actuating mechanism 860 and other working mechanisms.

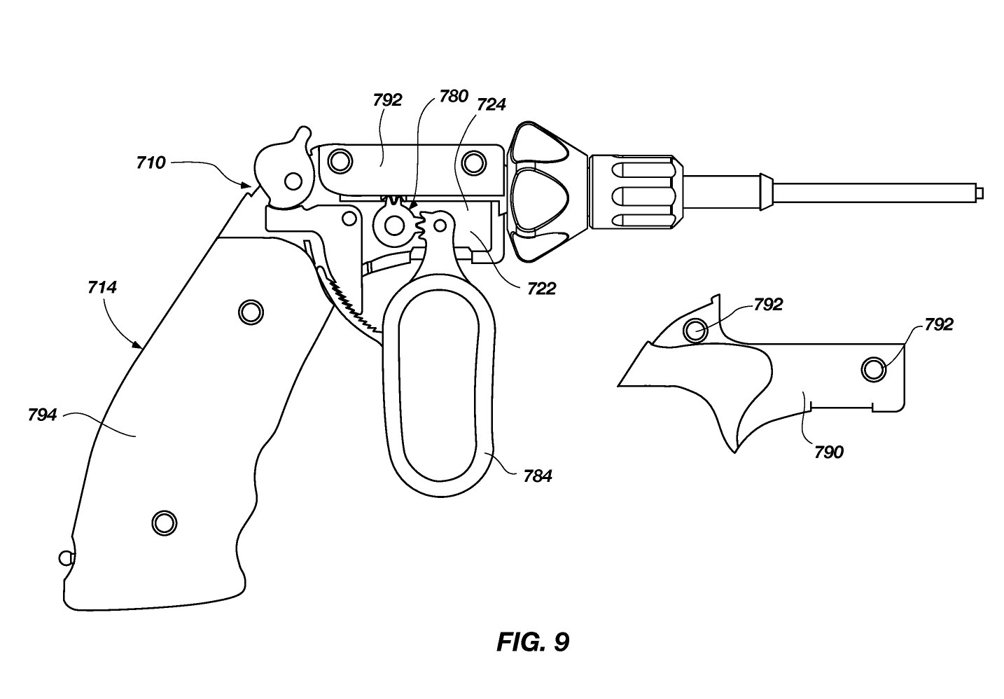

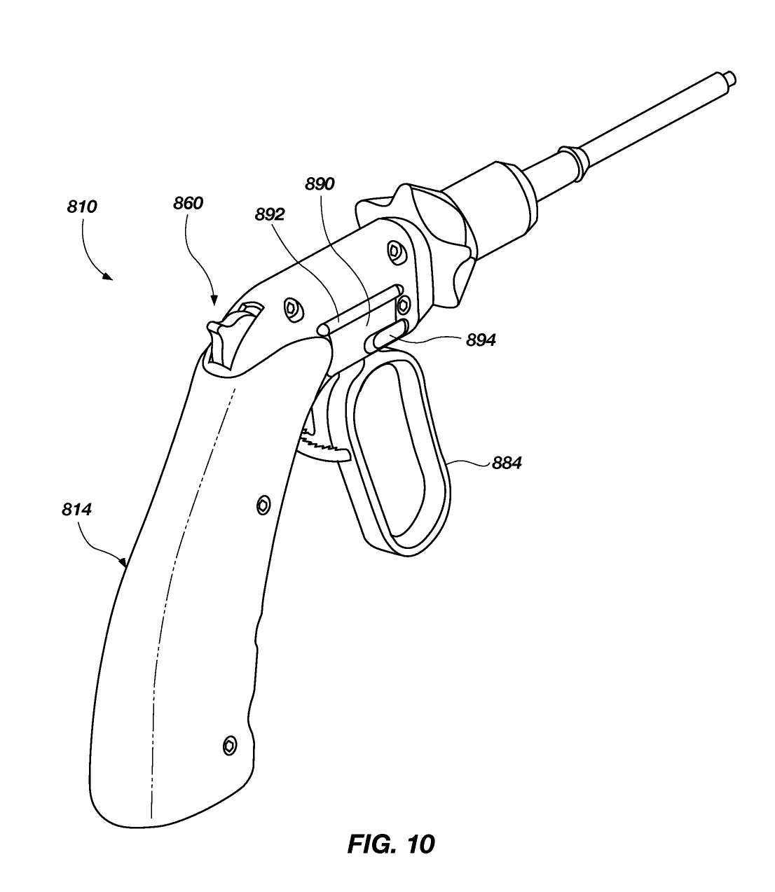

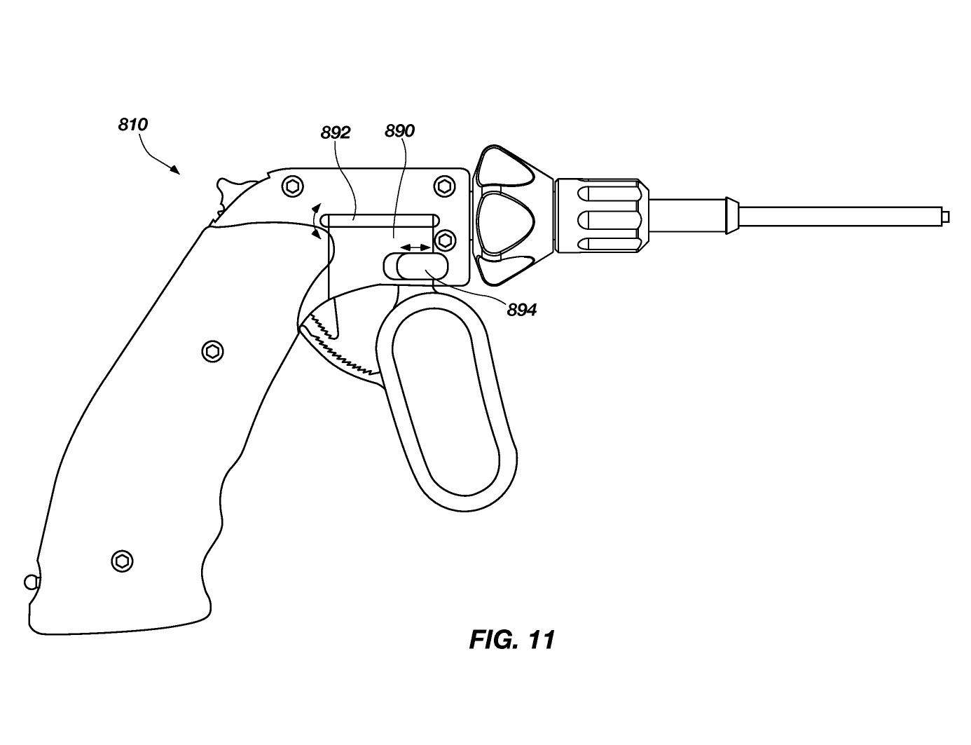

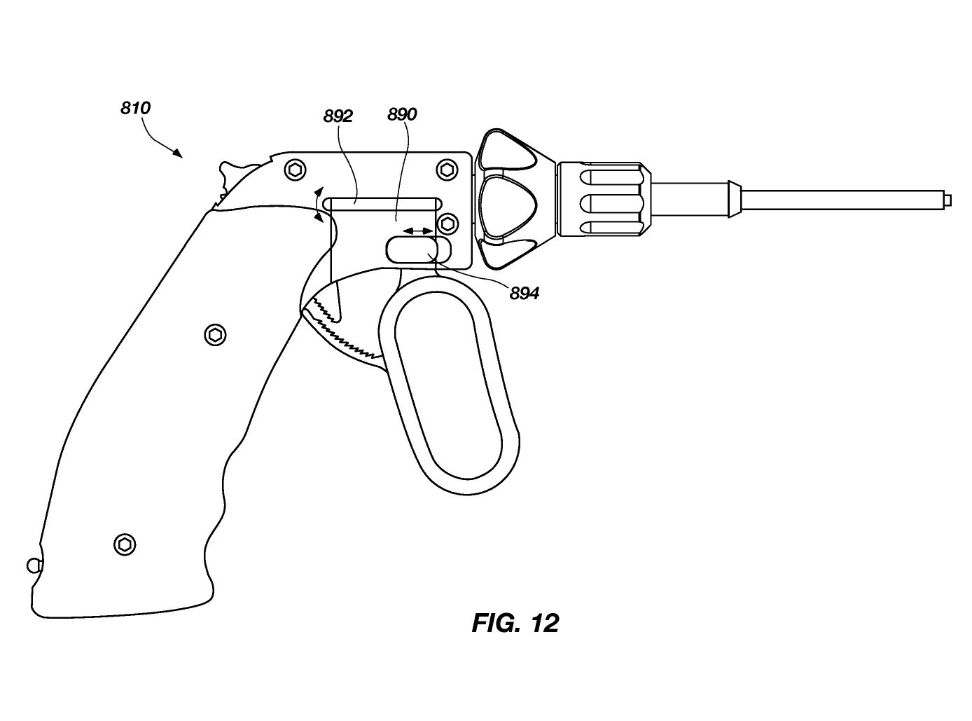

The hinged access panel 890 is secured in place and operable via a sliding lock 894 that is actuatable in a sliding manner back and forth to lock and unlock the access panel 890. The sliding lock 894 is operable about the surfaces of both a portion of the handle 814 and a portion of the access panel 890 to lock the access panel, as shown in FIG. 11. To unlock the access panel 890, it is slid in an opposite direction enough to release from the handle 814, and to allow the access panel 890 to pivot into an open position, as shown in FIG. 12. The hinged access panel 890 is an example of another access opening as a means for accessing the interior portion 822 of the handle 814.

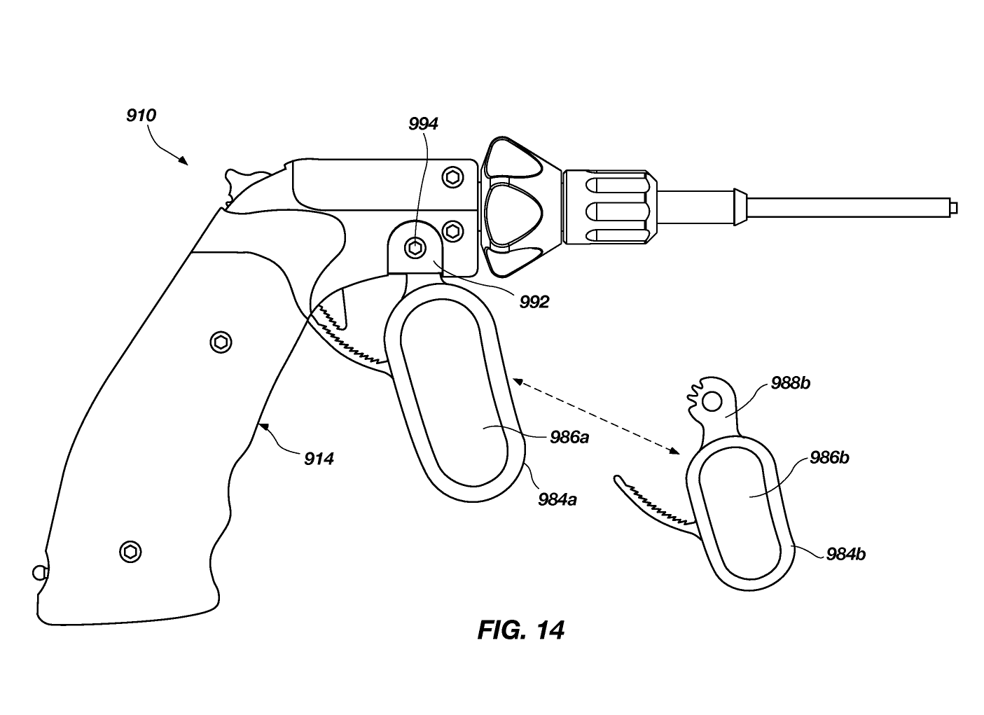

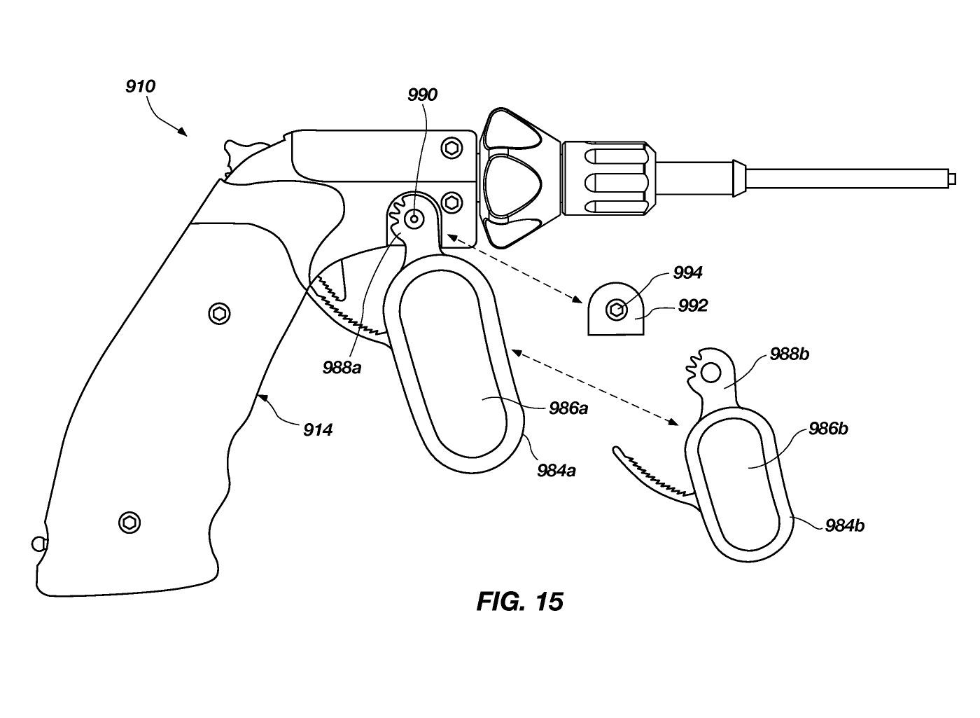

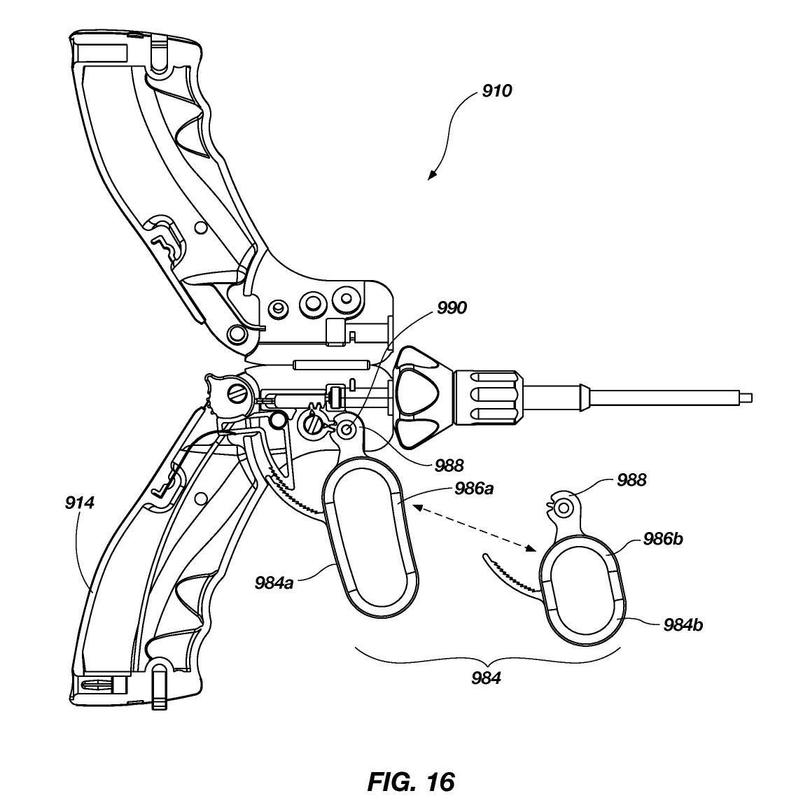

With reference to FIGS. 14-16, illustrated are various side views of a laparoscopic surgical instrument according to another exemplary embodiment of the present invention. In this embodiment, the laparoscopic surgical instrument 910 may comprise similar working mechanisms as described above, namely an actuation mechanism and a locking mechanism. As such, the description above is incorporated herein, where applicable. However, unlike any of the laparoscopic surgical instruments discussed above, the laparoscopic surgical instrument 910 comprises an ergonomic handle 914 operable with interchangeable trigger assemblies (such as first and second trigger assemblies 984a and 984b) so as to allow a surgeon to select a desired trigger configuration that is either needed or desired. Each of the interchangeable trigger assemblies 984a and 984b can be selectively removed or attached to the actuation mechanism so as to be operable with the actuation mechanism and about the handle 914.

In one aspect, the set of interchangeable trigger assemblies can include two triggers assemblies 984a and 984b, as shown in FIGS. 14-16. The first interchangeable trigger assembly 984a is pivotally supported and operable with the ergonomic handle 914 and the actuating mechanism. The first trigger assembly 984a comprises a loop 986a sized and shaped to receive a multiple fingers of the surgeon's hand, and also comprises an actuator 988a. The second trigger assembly 984b can also comprise a loop 986b sized and shaped to receive a single of finger of the surgeon's hand, and an actuator 988b. The first trigger assembly 984a and second trigger assembly 984b are designed to be interchangeable with one another. Of course, the specific trigger designs shown here are not meant to be limiting in any way. Indeed, other trigger designs may be configured that are also interchangeable.

As can be seen, the second trigger assembly 984b has a configuration different from the first trigger assembly 984a. In either case, the trigger assembly has a trigger or loop 986a or 986b that can receive at least one finger of the surgeon's hand and an actuator or gear 988 that facilitates operation of the actuating mechanism of the surgical instrument.

The first and second trigger assemblies 984a and 984b are shown as being releasably coupled to the handle 914 about a pivot point 990 and retained in place by way of a cap 992 and suitable fastener 994. Other coupling and retaining devices, as known in the art, can also be used to removably secure the first and second trigger assemblies 984a and 984b to the handle 914. For example, the triggers may be snapped into place using suitable structural elements supported by the handle to provide for this. Alternatively, fasteners or a press fit design may be implemented.

It is a particular advantage of the present invention laparoscopic surgical instrument that the trigger assemblies 984a and 984b can be interchanged with a different trigger assembly configuration. It will be appreciated that surgery requires precision and exactness in technique and that comfort of the surgeon's hand can affect the precision and accuracy of the surgery. Thus, the present invention advantageously allows the surgeon to choose a trigger that is most comfortable for the surgeon for a particular surgical procedure.

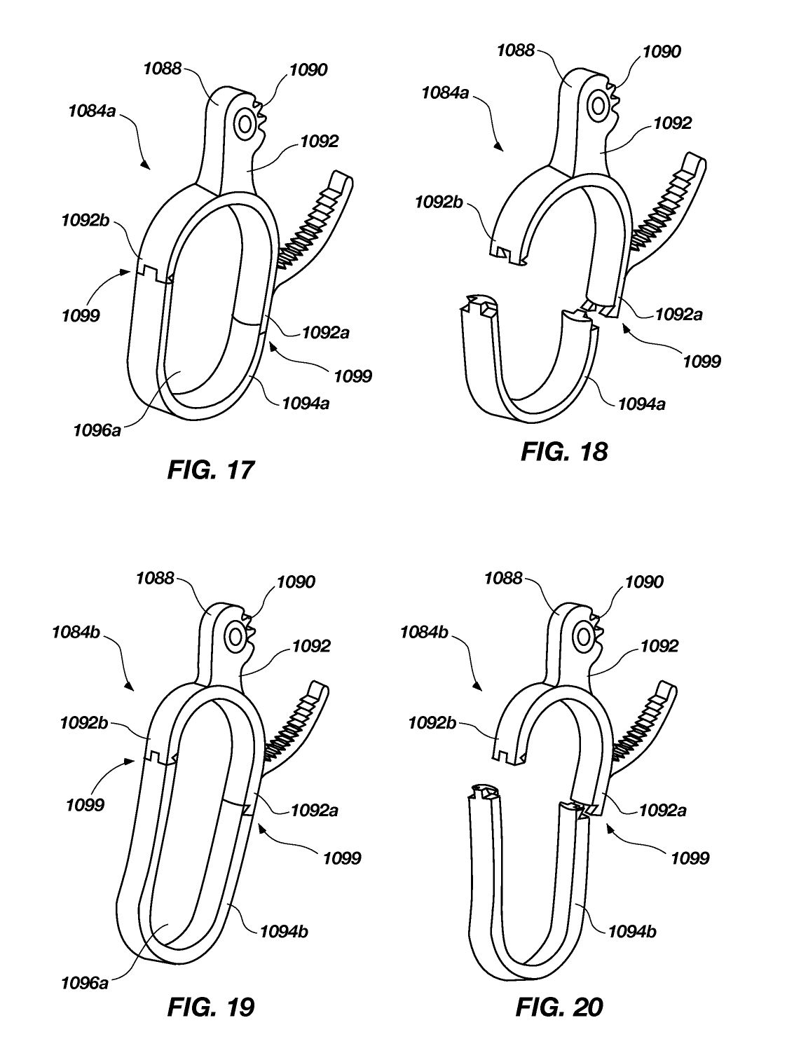

With reference to FIGS. 17-20, illustrated are perspective views of first and second trigger assemblies 1084a and 1084b, respectively, for the laparoscopic surgical instruments described above according to other exemplary embodiments of the present invention. FIGS. 17 and 18 illustrate a trigger assembly 1084a designed for a single finger of a user. FIGS. 19 and 20 illustrate a trigger assembly 1084b designed to receive multiple fingers of a user. The trigger assemblies 1084a and 1084b share a common upper portion having an actuator 1088 and end attachments 1092a and 1092b, with the lower portions 1094a and 1094b being interchangeable.

As shown, the trigger assemblies 1084a and 1084b can include an actuator 1088 that is common between the two trigger assemblies 1084a and 1084b. The actuator 1088 can include a trigger gear 1090 and at least one attachment end 1092 coupled to the trigger gear 1090. As illustrated, the actuator 1088 can include two attachment ends 1092a and 1092b that can be spaced apart from one another and that can form an upper portion of the trigger assemblies. The two attachment ends 1092a and 1092b can include tongue and groove, snap-lock type attachments that can secure an interchangeable portion to the attachment ends to complete the trigger assemblies.

The interchangeable portions that form the trigger assemblies 1084a and 1084b can include trigger handles 1094a and 1094b, each being configured differently, and that comprise end portions that can attach to the attachment ends 1092a and 1092b of the actuator 1088. The trigger handles 1094a and 1094b can each have a different size and shape such that each trigger handle forms a finger placement configuration that is different from the other interchangeable trigger handles. In this way, the first trigger handle 1094a of the trigger assembly can be removed from the actuator 1088 and can be replaced with the second trigger handle 1094b in order to accommodate the preference of the surgeon using the laparoscopic instrument, or a particular surgical technique that may be easier to perform with a particular trigger handle configuration.

For example, as shown in FIGS. 17 and 18, the first trigger handle 1094a can be shaped to form a loop 1096a that is sized to fit a single finger of the surgeon. Additionally, as shown in FIGS. 19 and 20, the second trigger handle 1094b can be shaped to form an ovalized loop 1096b that is sized to fit at least two fingers of the surgeon. Other sizes and shaped for trigger handles, as known in the art, can also be used as the trigger handle for the laparoscopic surgical instrument of the present invention.

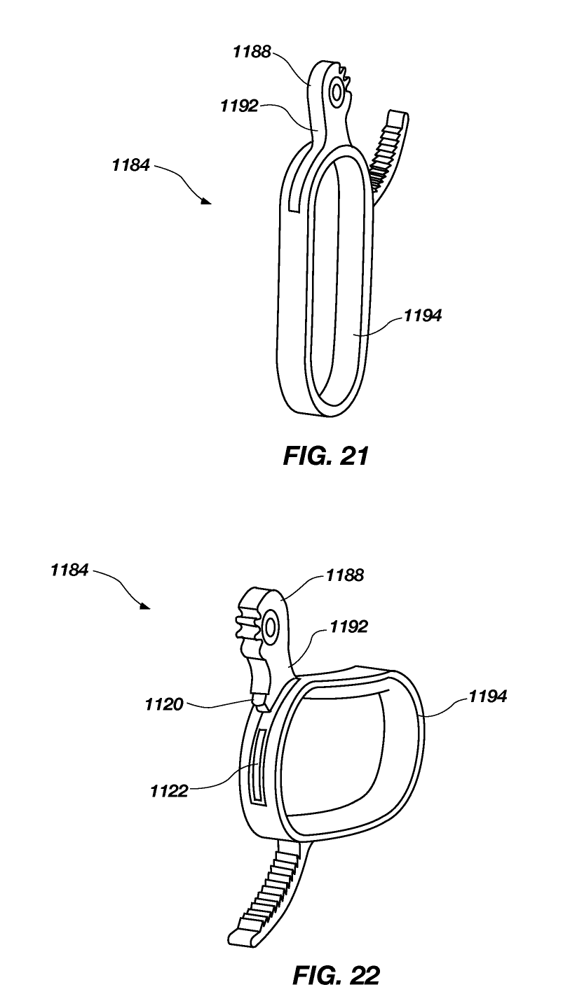

With reference to FIGS. 21-22, illustrated are perspective views of another removable trigger assembly 1184 for the laparoscopic surgical instrument discussed above according to another exemplary embodiment of the present invention. In this case, the actuator 1188 has a single attachment end 1192 that can be removably coupled to a variety of trigger handles 1194. The attachment end 1192 can include an arcuate T shaped flange 1120 that can fit into a T shaped slot 1122 on a corresponding trigger handle 1194 to removably secure the trigger handle 1194 to the actuator 1188 to form the trigger assembly 1184. Multiple different types and styles of triggers may be used as needed or desired. FIG. 21 illustrates the actuator with its single attachment end 1192 removably coupled to the trigger handle 1194, with the T-shaped flange 1120 received within the corresponding T-shaped slot 1122. FIG. 22 illustrates these two components separated from one another in anticipation of a different trigger handle (not shown).

Referring back to FIG. 7-A, the laparoscopic surgical instrument 510 further comprises interchangeable triggers, namely triggers 584a and 584b that may be selectively removed and associated with the actuating mechanism 580. In this particular embodiment, the interchangeable triggers may be removably secured to the handle 514 about the pivot point 596 (as provided by a post, for example) and caused to be engaged with the actuating mechanism upon opening the handle 514 (e.g., pivoting the first handle portion 514a away from the second handle portion 514b as described above) to expose the interior portion 522 of the handle 514.

While several different connection configurations are shown herein to provide interchangeable triggers or trigger assemblies, these are not meant to be limiting in any way as one skilled in the art may realize other ways to provide for interchangeable triggers or trigger assemblies.

The present invention also provides for a method for cleaning and sterilizing of a surgical instrument including obtaining a laparoscopic surgical instrument configured to be ergonomic and anthropometrically correct. The laparoscopic surgical instrument has an ergonomic handle with a wall structure defining an interior portion adapted to contain at least a portion of one or more working mechanisms. The interior portion of the handle can be accessed to expose one or more surfaces of the interior and at least a portion of each of the working mechanisms. The exposed surfaces and said working mechanisms can be cleaned and sterilized to prepare the surgical instrument for subsequent use.

The present invention also provides for a method for manufacturing a surgical instrument including providing a handle having a wall structure defining an interior portion, and adapted to contain at least a portion of one or more working mechanisms. The one or more working mechanisms are supported about the handle. Access to the one or more working mechanisms and one or more surfaces of the interior portion is facilitated through one or more access openings formed in the handle.

Additional embodiments are shown in Appendix A attached hereto which is incorporated by reference in its entirety for all purposes.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

More specifically, while illustrative exemplary embodiments of the invention have been described herein, the present invention is not limited to these embodiments, but includes any and all embodiments having modifications, omissions, combinations (e.g., of aspects across various embodiments), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function limitation are expressly recited in the description herein. Accordingly, the scope of the invention should be determined solely by the appended claims and their legal equivalents, rather than by the descriptions and examples given above.

What is claimed and desired to be secured by Letters Patent is:

1. A laparoscopic surgical instrument configured to be ergonomic and anthropometrically correct, said laparoscopic surgical instrument comprising:
an ergonomic handle configured to orient a hand of a surgeon in a functional position, said handle comprising a wall structure defining an interior portion, and adapted to contain at least a portion of one or more working mechanisms;
an actuating mechanism actuatable by said surgeon and at least partially supported within said interior portion of said handle;
a working shaft having a proximal end coupled to and operable with said actuating mechanism, said working shaft having an elongate configuration and a distal working end configured to couple a surgical tool to be manipulated by said surgeon; and
means for accessing said interior portion of said handle to expose an inner side of said wall structure and at least a portion of each of said working mechanisms for cleaning, sterilization and maintenance purposes.

2. The laparoscopic surgical instrument of claim 1, wherein said means for accessing comprises:
a two-piece handle configuration, wherein a first handle portion is operable with a second handle portion to define said handle, said first and second handle portions being separable from one another to provide access to said interior portion; and
a hinge operable to interrelate said first and second handle portions, and to facilitate separation, alignment and union of said first and second handle portions.

3. The laparoscopic surgical instrument of claim 1, wherein said means for accessing comprises:
a removable access panel removably coupled to said wall structure of said ergonomic handle, said access panel being removable from said wall structure to provide access to said interior portion containing said actuating mechanism and said working mechanisms.

4. The laparoscopic surgical instrument of claim 1, wherein said means for accessing comprises:
a hinged access panel hingedly coupled to said wall structure of said ergonomic handle adjacent said actuating mechanism, said access panel being pivotal about said hinge to provide access to said interior portion containing said actuating mechanism and said working mechanisms.

5. The laparoscopic surgical instrument of claim 1, wherein said means for accessing comprises an access opening defined by a boundary of said handle.

6. The laparoscopic surgical instrument of claim 1, wherein said one or more working mechanisms are selected from the group consisting of an actuating mechanism, a locking mechanism, a working shaft, a conducting rod, a gear system, an actuator shaft, a trigger, a trigger hinge, and combinations thereof.

7. The laparoscopic surgical instrument of claim 1, further comprising a first interchangeable trigger assembly pivotally supported and operable with said ergonomic handle and said actuating mechanism, said first trigger assembly being selectively interchangeable with a second trigger assembly having a configuration different from said first trigger assembly.

8. The laparoscopic surgical instrument of claim 1, wherein said actuating mechanism further comprises an actuator shaft and a gear operable to displace said actuator shaft with a mechanical advantage upon actuation of a trigger assembly pivotally coupled to said ergonomic handle and operable with said actuating mechanism when said trigger assembly is rotated by said surgeon.

9. The laparoscopic surgical instrument of claim 1, further comprising a locking mechanism that directly engages and locks said trigger assembly in one of a plurality of positions, said locking mechanism comprising a release located in an anthropometrically correct position.

10. The laparoscopic surgical instrument of claim 9, wherein said locking mechanism comprises:
a first link operable with said trigger and having a plurality of teeth formed on at least a portion of said first link;
a second link pivotally mounted to said handle and having at least one tooth formed on at least a portion of said second link, and configured to engage said plurality of teeth on said first link to lock said trigger assembly in one of said plurality of positions, said first and second links configured to provide a ratcheting function, wherein said actuating mechanism is allowed to move freely in one direction, while being restricted to move in an opposite direction;
a biasing element configured to bias said second link about a pivot point and to an engaged position with respect to said plurality of teeth; and
a thumb actuated release supported by said handle and configured to engage said second link to overcome said biasing element to disengage said second link from said plurality of teeth of said first link, thus enabling said trigger assembly to move in said opposite direction.

11. The laparoscopic surgical instrument of claim 10, wherein said second link comprises a first end and a second end, said first end having said at least one tooth formed thereon, and said second end oriented substantially perpendicular to said first end and extending rearward toward said thumb actuated release.

12. The laparoscopic surgical instrument of claim 11, wherein said release is rotatably mounted about a pivot point of said ergonomic handle, said release comprising an eccentric actuator configured to engage said second end of said second link, wherein by rotating said release in a given direction said eccentric actuator engages said second end of said second link to pivot said second link about said pivot point and overcome said biasing element, thus causing said second link to disengage from said plurality of teeth of said first link, thereby allowing said trigger assembly to freely move in any direction.

13. The laparoscopic surgical instrument of claim 12, wherein said release is located ergonomically and anthropometrically, thus enabling said surgeon to operate said release and said trigger and said actuating mechanism simultaneously with the same hand.

14. The laparoscopic surgical instrument of claim 1, further comprising:
a trigger assembly pivotally supported and operable with said ergonomic handle and said actuating mechanism, said trigger assembly extending from said interior space through a trigger aperture in said wall structure;
a trigger seal disposed around said trigger aperture and said trigger to seal said interior space from contamination;
a locking mechanism that directly engages and locks said trigger assembly in one of a plurality of positions, said locking mechanism comprising a release located in an anthropometrically correct position on said ergonomic handle and extending through a release aperture in said wall structure; and
a release seal disposed around said release aperture and said release to seal said interior space from contamination.

15. The laparoscopic surgical instrument of claim 1, further comprising a drain hole disposed in said ergonomic handle to allow cleaning fluids to escape said interior space during cleaning and sterilization of said ergonomic handle.

16. A laparoscopic surgical instrument configured to be ergonomic and anthropometrically correct, said laparoscopic surgical instrument comprising:
an ergonomic handle configured to orient a hand of a surgeon in a functional position, said handle comprising a wall structure defining an interior portion, and adapted to contain at least a portion of one or more working mechanisms;
an actuating mechanism actuatable by said surgeon and supported within said interior portion of said handle;
a trigger assembly pivotally supported and operable with said ergonomic handle and said actuating mechanism, said trigger assembly comprising a trigger configured to receive at least one finger of said surgeon and an actuator that operates said actuating mechanism;
a working shaft having a proximal end coupled to and operable with said actuating mechanism, said working shaft having an elongate configuration and a distal working end configured to couple a surgical tool to be manipulated by said surgeon; and
a locking mechanism that directly engages and locks said trigger assembly in one of a plurality of positions, said locking mechanism comprising a release located in an anthropometrically correct position.

17. A laparoscopic surgical instrument configured to be ergonomic and anthropometrically correct, said laparoscopic surgical instrument comprising:
a handle operable with one or more working mechanisms;
an actuating mechanism actuatable by said surgeon and supported by said handle;
a working shaft having a proximal end coupled to and operable with said actuating mechanism, said working shaft having an elongate configuration and a distal working end configured to couple a surgical tool to be manipulated by said surgeon; and
a first trigger assembly pivotally supported by said handle and operable with said actuating mechanism, said first trigger assembly being selectively interchangeable with a second trigger assembly having a configuration different from said first trigger assembly.

18. The laparoscopic surgical instrument of claim 17, wherein said first and second trigger assemblies each comprise a trigger configured to receive at least one finger of said surgeon and an actuator that operates said actuating mechanism.

19. The laparoscopic surgical instrument of claim 17, wherein said first and second trigger assemblies each comprise a removable trigger handle coupleable to an actuator that operates said actuating mechanism, said removable trigger handle of said first trigger having a finger placement configuration different from said second trigger assembly.

20. A method for cleaning and sterilizing a surgical instrument, said method comprising:
obtaining a laparoscopic surgical instrument having a handle with a wall structure defining an interior portion adapted to contain at least a portion of one or more working mechanisms;
accessing said interior portion of said handle to expose one or more surfaces of said interior and at least a portion of each of said working mechanisms; and
cleaning and sterilizing said exposed surfaces and said working mechanisms to prepare said surgical instrument for subsequent use.
